(19) United States
(12) Patent Application Publication
(10) Pub. No.: US 2025/0375531 A1
(43) Pub. Date: Dec. 11, 2025

(54) TREATMENT OF HR+/HER2- BREAST CANCER USING SACITUZUMAB GOVITECAN

(71) Applicant: Gilead Sciences, Inc., Foster City, CA (US)

(72) Inventors: See-Chun Phan, Menlo Park, CA (US); Wendy J. Verret, Mill Valley, CA (US)

(21) Appl. No.: 19/255,499

(22) Filed: Jun. 30, 2025

Related U.S. Application Data
(63) Continuation of application No. 18/671,393, filed on May 22, 2024.
(60) Provisional application No. 63/503,882, filed on May 23, 2023.

(51) Int. Cl. A61K 47/68; A61K 9/00; A61P 35/00

(52) U.S. Cl. CPC A61K 47/6851; A61K 9/0019; A61K 47/68037; A61P 35/00

(57) ABSTRACT

The present disclosure relates to methods of treating a HR+/HER2- breast cancer in a human subject, wherein the subject has received previously endocrine-based therapy and at least two additional systemic therapies, comprising administering a pharmaceutical composition comprising SG.

### CROSS-REFERENCES TO RELATED APPLICATIONS

This application is a continuation of U.S. patent application Ser. No. 18/671,393, filed May 22, 2024, which claims the benefit of priority to U.S. Provisional Patent Application No. 63/503,882, filed on May 23, 2023, and titled “TREATMENT OF HR+/HER2- BREAST CANCER USING SACITUZUMAB GOVITECAN,” the contents of each of which are incorporated by reference herein.

### SEQUENCE LISTING

The instant application contains a Sequence Listing, which has been submitted via Patent Center. The Sequence Listing titled 210196-323003US_SL.xml, which was created on Jun. 25, 2025 and is 12,526 bytes in size, is hereby incorporated by reference in its entirety.

### FIELD

The present disclosure relates to methods of treatment of HR+/HER2- breast cancer using sacituzumab govitecan (“SG”).

### BACKGROUND

Breast cancer is the second leading cause of death in women, and approximately 70% of breast cancers are hormone receptor positive/human epidermal growth factor receptor 2-negative (“HR+/HER2-”) (National Cancer Institute. cancer stat facts: female breast cancer subtypes).

Initial treatments for these patients are endocrine-based therapies, including combinations with targeted therapies, such as cyclin-dependent kinase (CDK) 4/6, mammalian target of rapamycin (mTOR), and phosphatidylinositol-3-kinase inhibitors. Treatment options for endocrine-resistant/treatment-refractory disease include single-agent chemotherapy. However, response rates to later-line therapies are low. In summary, given the poor outcomes (limited effectiveness and poor tolerability) and limited remaining treatment options available, there remains a high unmet need for patients with HR+/HER2-breast cancer who have received prior endocrine therapy and chemotherapy for locally advanced or metastatic disease. Prolonging overall survival (“OS”), improving efficacy, and maintaining or improving quality of life (“QOL”) with manageable toxicities continues to represent an area of unmet medical need in HR+/HER2- metastatic breast cancer.

Sacituzumab govitecan (“SG”) is a first-in-class antibody-drug-conjugate (“ADC”) comprised of a humanized monoclonal antibody (“hRS7”) that binds to the cell-surface receptor, Trop 2. a payload (“SN-38”) that is a topoisomerase I inhibitor, and a linker (“CL2A”), that couples the antibody to the payload. There is a high expression of Trop 2 in breast cancers, such as the HR+/HER2- subtype.

The high expression of Trop 2 in breast cancers, coupled to the high unmet need led to the design of a phase 1/2, single arm clinical trial (NCT01631552), open to those whose disease had previously progressed on endocrine-based therapy and at least one prior chemotherapy for metastatic breast cancer. Preliminary data from this study was used to inform the design of a phase 3 study of SG versus treatment of physician's choice (TPC) in subjects with HR+/HER2- breast cancer that is refractory or relapsed after at least 2, and no more than 4, prior systemic chemotherapy regimens including: at least one prior anticancer hormonal treatment and at least one cyclin-dependent kinase inhibitor 4/6 treatment.

In October 2022, the FDA granted priority review for the supplemental biologics application for the use of SG for the treatment of HR+/HER2- breast cancer based on meeting a key secondary endpoint of overall survival over comparator chemotherapy. The FDA grants priority review for therapies that, if approved, would be significant improvements to the safety or effectiveness of treatment, diagnosis or prevention of serious conditions when compared to standard applications. On Feb. 3, 2023, the FDA approved SG for treatment of patients with unresectable locally advanced or metastatic HR+/HER2- breast cancer who have received endocrine-based therapy and at least two additional therapies in the metastatic setting (e.g., SG's label updated 02/2023). SG is marketed under the name TRODELVY.

### SUMMARY

In certain embodiments, disclosed herein are methods of treating a HR+/HER2- breast cancer in a human subject, wherein the subject previously received endocrine-based therapy and at least two additional systemic therapies, comprising administering to the subject a dosage of 10 mg/kg of SG as an intravenous infusion once weekly on day 1 and day 8 of one or more 21-day treatment cycles, and wherein the method achieves an overall survival (OS) in the subject of at least 14 months from initiation of treatment with SG.

In certain embodiments, the methods disclosed herein achieve an OS in the subject of 14.5 months.

In certain embodiments, the endocrine-based therapy comprises CDK4/CDK 6 inhibitor therapy.

In certain embodiments, at least one of the additional systemic therapies was administered in the metastatic setting. In certain embodiments. at least one of the at least two additional systemic therapies is administered in the metastatic setting.

In certain embodiments, at least one of the at least two additional systemic therapies comprises chemotherapy.

In certain embodiments, chemotherapy comprises a taxane.

In certain embodiments. the subject previously received at least two, but no more than four. additional systemic therapies.

In certain embodiments. the methods comprising administering SG as disclosed herein achieve a partial response in the subject. In certain embodiments. the methods comprising administering SG as disclosed herein achieve a complete response in the subject.

In certain embodiments. the methods comprising administering SG as disclosed herein achieve a progression-free survival (PFS) of at least 5 months in the subject from initiation of treatment with SG. In certain embodiments. the methods comprising administering SG as disclosed herein achieve a PFS of 5.5 months in the subject from initiation of treatment with SG. In certain embodiments. the methods comprising administering SG as disclosed herein achieve a PFS of 5.5 months and an OS of 14.5 months at a follow-up of about 12.75 months from initiation of treatment with SG.

In certain embodiments, when the method is used to treat HR+/HER2- breast cancer in a population of human subjects, the method achieves a median OS of 14.5 months and a median PFS of 5.5 months at a median follow-up of about 12.75 months from initiation of treatment with SG.

In certain embodiments. the subject treated using the methods as disclosed herein received prior therapy in the locally advanced or metastatic setting. In certain embodiments. the subject treated using the methods as disclosed herein received prior therapy in the metastatic setting.

In certain embodiments. the dosage of SG as disclosed herein is modified or interrupted due to neutropenia.

In certain embodiments. the subject treated using the methods as disclosed herein is homozygous or heterozygous for a UGT1A1*28 allele.

In certain embodiments. the dosage of SG as disclosed herein is reduced by 25% after a first occurrence of severe neutropenia and granulocyte-colony stimulating factor (G-CSF) is administered. In certain embodiments. the dosage of SG is reduced by 50% after a second occurrence of severe neutropenia and G-CSF is administered. In certain embodiments. administration of SG is discontinued after a third occurrence of severe neutropenia and G-CSF is administered. In certain embodiments. the dosage of SG is modified or interrupted due to severe non-neutropenic toxicity.

In certain embodiments, the HR+/HER2- breast cancer is HR+/HER2- low breast cancer or HR+/HER2-IHC0 breast cancer.

In certain embodiments. disclosed herein are methods of treating a HR+/HER2- breast cancer in a human subject, wherein the breast cancer is a HR+/HER2-low breast cancer. and wherein the subject previously received endocrine-based therapy and at least two additional systemic therapies. comprising administering to the subject a dosage of 10 mg/kg of SG as an intravenous infusion once weekly on day 1 and day 8 of one or more 21-day treatment cycles. and wherein the method achieves an OS in the subject of at least 15 months from initiation of treatment with SG. In certain embodiments. the method achieves an OS of 15.4 months from initiation of treatment with SG. In certain embodiments. the method achieves an OS of 15.4 months, at a follow-up of about 12.75 months from initiation of treatment with SG.

In certain embodiments. disclosed herein are methods of treating a HR+/HER2- breast cancer in a human subject. wherein the subject has HR+/HER2- IHC0 breast cancer. and wherein the subject previously received endocrine-based therapy and at least two additional systemic therapies. the method comprising administering to the subject a dosage of 10 mg/kg of SG as intravenous infusion once weekly on day 1 and day 8 of one or more 21-day treatment cycles. wherein the method achieves an OS in the subject of at least 13 months from initiation of treatment with SG. In certain embodiments. the method achieves an OS in the subject of 13.6 months from initiation of treatment with SG. In certain embodiments. the method achieves an OS of 13.6 months at a follow-up of about 12.75 months from initiation of treatment with SG.

In certain embodiments. disclosed herein is a method of treating a HR+/HER2- breast cancer in a human subject that is statistically significantly superior to a treatment of physician's choice (TPC), wherein the subject previously received an endocrine-based therapy and at least two additional systemic therapies. comprising administering to the subject a dosage of 10 mg/kg of SG as an intravenous infusion once weekly on day I and day 8 of one or more 21-day treatment cycles. and wherein when the method is used to treat HR+/HER2- breast cancer in a population of human subjects. the population of subjects treated with SG achieves a statistically significantly greater median OS and median PFS compared to a control group of human subjects treated with the TPC.

In certain embodiments. disclosed herein is a method of treating HR+/HER2- breast cancer in a population of human subjects having HR+/HER2- breast cancer, that is statistically significantly superior to a treatment of physician's choice (TPC), wherein the subjects previously received endocrine-based therapy and at least two additional systemic therapies. wherein when a dosage of 10 mg/kg of SG is administered as an intravenous infusion once weekly on day 1 and day 8 of one or more 21-day treatment cycles. the method achieves a statistically significantly greater median OS and median PFS compared to a control group of human subjects treated with the TPC.

In certain embodiments. the method achieves a median OS of 14.5 months in the population of human subjects treated with SG and a median OS of 11.2 months in the control group of human subjects treated with the TPC at a median follow-up of about 12.75 months. In certain embodiments. the method achieves a median PFS of 5.5 months in the population of human subjects treated with SG and a median PFS of 4.0 months in the control group of human subjects treated with TPC at a median follow-up of about 12.75 months.

In certain embodiments, disclosed herein are methods of achieving an OS of at least 14 months in a human subject having HR+/HER2- breast cancer comprising administering a dosage of 10 mg/kg of SG is as an intravenous infusion on day 1 and day 8 of one or more 21-day treatment cycles. wherein the method achieves an OS of at least 14 months from initiation of treatment with SG.

In certain embodiments, disclosed herein is a method of achieving a median OS of 14.5 months in the population of human subjects treated with SG and a median OS of 11.2 months in the control group of human subjects treated with the TPC at a median follow-up of about 12.75 months. In certain embodiments. the method as disclosed herein achieves a median PFS of 5.5 months in the population of human subjects treated with SG and a median PFS of 4.0 months in the control group of human subjects treated with the TPC at a median follow-up of about 12.75 months.

In certain embodiments, disclosed herein is a method of achieving a clinically meaningful benefit in a human subject having HR+/HER2- breast cancer. comprising administering a dosage of 10 mg/kg of SG to the subject as an intravenous infusion once weekly on day I and day 8 of one or more 21-day treatment cycles.

In certain embodiments, disclosed herein is a method of statistically significant treatment of HR+/HER2- breast cancer in human subjects who previously received an endocrine-based therapy and at least two additional systemic therapies. comprising administering to each said subject a dosage of 10 mg/kg of SG on day I and day 8 of one or more 21-day treatment cycles. wherein said treatment achieves a statistically significant clinical response compared to a control group of human subjects treated with the TPC. In certain embodiments, the statistically significant clinical response is median OS. In certain embodiments, the statistically significant clinical response is median PFS. In certain embodiments. the statistically significant clinical response is median duration of response (DOR). In certain embodiments. the statistically significant clinical response is overall response rate (ORR). In certain embodiments. the statistically significant clinical response is confirmed best overall response (CBR).

In certain embodiments. the human subjects are treated with the methods as disclosed herein until death or unacceptable toxicity.

In certain embodiments. disclosed herein are methods of treating a HR+/HER2- breast cancer in a human subject. wherein the subject previously received CDK4/CDK6 inhibitor therapy. taxane therapy. and at least two, but more than four. additional systemic therapies. comprising administering to the subject a dosage of 10 mg/kg of SG on day 1 and day 8 of one or more 21-day treatment cycles, wherein said treatment achieves a statistically significant clinical response in the subject. In certain embodiments, at least one of the systemic therapies is administered in the metastatic setting.

In certain embodiments, disclosed herein is a method of increasing overall survival of a patient having HR+/HER2- breast cancer. The method comprises administering to a patient in need thereof: 10 mg/kg SG as an intravenous infusion once weekly on day 1 and day 8 of one or more 21-day treatment cycles, wherein the patient has HR+/HER2-breast cancer and has previously received an endocrine-based therapy and at least two additional systemic therapies.

In certain embodiments, the method comprises administering SG for about 8 cycles or more. In certain embodiments. the method comprises administering SG for about 11 cycles or more. In certain embodiments. the patient has HR+/HER2-IHC0 breast cancer. In certain embodiments. the patient has HR+/HER2low breast cancer.

In certain embodiments. disclosed herein is a method of treating a HR+/HER2- breast cancer in a patient. wherein the patient previously received endocrine-based therapy and at least two additional systemic therapies. comprising administering to the patient in need thereof 10 mg/kg SG as an intravenous infusion once weekly on day I and day 8 of one or more 21-day treatment cycles. and achieving an overall survival in the patient of at least about 14 months from initiation of treatment with SG. In certain embodiments. comprising administering to the patient in need thereof 10 mg/kg SG as an intravenous infusion once weekly on day 1 and day 8 of at least (19) 21-day treatment cycles.

In certain embodiments. disclosed herein is a method of treating a HR+/HER2- breast cancer in a patient. wherein the breast cancer has a Trop 2 expression of H-score<100, wherein the patient previously received endocrine-based therapy and at least two additional systemic therapies. comprising administering to the patient in need thereof 10 mg/kg as an intravenous infusion once weekly on day 1 and day 8 of at least about (19) 21-day treatment cycles: and achieving an overall survival in the patient of at least about 14 months from initiation of treatment with SG.

In certain embodiments, disclosed herein is a method of increasing overall survival of a patient having HR+/HER2- breast cancer. The method comprises administering to a patient in need thereof: SG as an intravenous infusion once weekly on day 1 and day 8 of one or more 21-day treatment cycles, wherein the patient has HR+/HER2- breast cancer and has previously received an endocrine-based therapy and at least two additional systemic therapies.

In certain embodiments, the method comprises administering SG for about 8 cycles or more. In certain embodiments, the method comprises administering SG for about 11 cycles or more. In certain embodiments, the patient has HR+/HER2-IHC0 breast cancer. In certain embodiments, the patient has HR+/HER2low breast cancer.

### BRIEF DESCRIPTION OF THE DRAWINGS

FIG. 1 shows the design of a Phase 3 clinical trial (NCT03901339). The clinical trial is referred to as “TROPiCS-02” or “TROPiCS-02.”

FIG. 2 is a Kaplan-Meier Plot showing final PFS results from the TROPiCS-02 clinical trial.

FIG. 3 is a Kaplan-Meir Plot showing the final OS results from the TROPiCS-02 clinical trial.

FIG. 4 is a Kaplan-Meir Plot showing final PFS results from the HR+/HER2-low subgroup from the TROPiCS-02 clinical trial.

FIG. 5 is a Kaplan-Meir Plot showing final PFS results of the HR+/HER2-IHCO subgroup from the TROPiCS-02 clinical trial.

FIG. 6 is a Kaplan-Meir Plot showing the final OS results of the HR+/HER2-low subgroup from the TROPiCS-02 clinical trial.

FIG. 7 is a Kaplan-Meir Plot showing the final OS results of the HR+/HER2-IHCO subgroup from the TROPiCS-02 clinical trial.

FIGS. 8a and 8b show the sequences of the heavy (FIG. 8a) and the light (FIG. 8b) chains of the antibody (hRS7) in SG.

FIG. 9 is a chart showing the hierarchical testing procedures in the TROPiCS-02 clinical trial.

FIG. 10 shows the final PFS results from the TROPiCS-02 clinical trial by subgroup.

FIG. 11 shows the final OS results from the TROPiCS-02 clinical trial by subgroup.

FIG. 12 shows the final response data from the TROPiCS-02 clinical trial.

FIG. 13 shows PFS by Trop-2 expression level from the TROPiCS-02 clinical trial. FIG. 13A shows PFS for subjects with an H-score<100; and FIG. 13B shows PFS for subjects with an H-score≥100.

FIG. 14 shows OS by Trop-2 expression level from the TROPiCS-02 clinical trial. FIG. 14A shows PFS for subjects with an H-score<100; and FIG. 14B shows PFS for subjects with an H-score≥100.

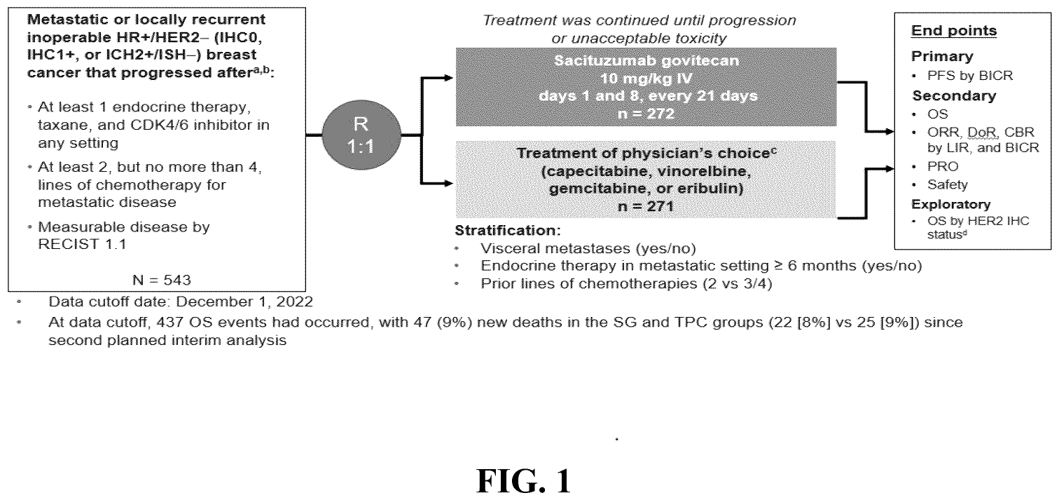

### DETAILED DESCRIPTION

### Anti-TROP2 ADC: Sacituzumab govitecan (“SG”)

SG is a pharmaceutical composition comprising an antibody-drug conjugate (“ADC”) comprised of (1) a drug (“SN-38”), a topoisomerase 1 inhibitor that is an active metabolite of irinotecan: (2) a linker (“CL2A”); and a humanized monoclonal antibody (“hRS7 IgG.” or “sacituzumab”). CL2A couples SN-38 to hRS7, which binds to Trop-2.

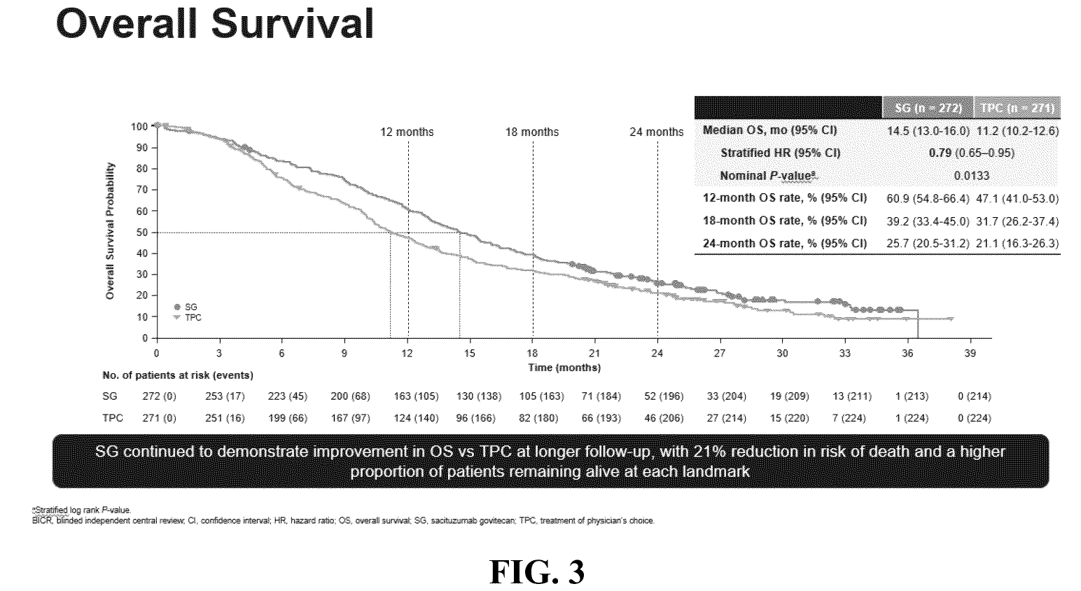

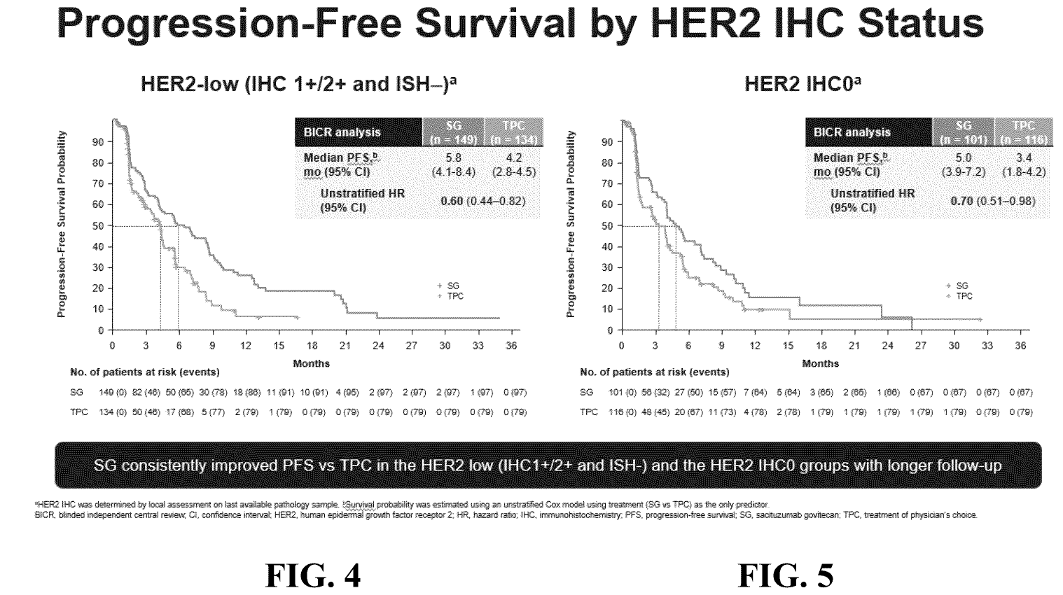

In certain embodiments, hRS7 is described, e.g., in WO2003074566, FIGS. 3 and 4, incorporated by reference in its entirety.

In certain embodiments, SG is represented by Formula I as shown below.

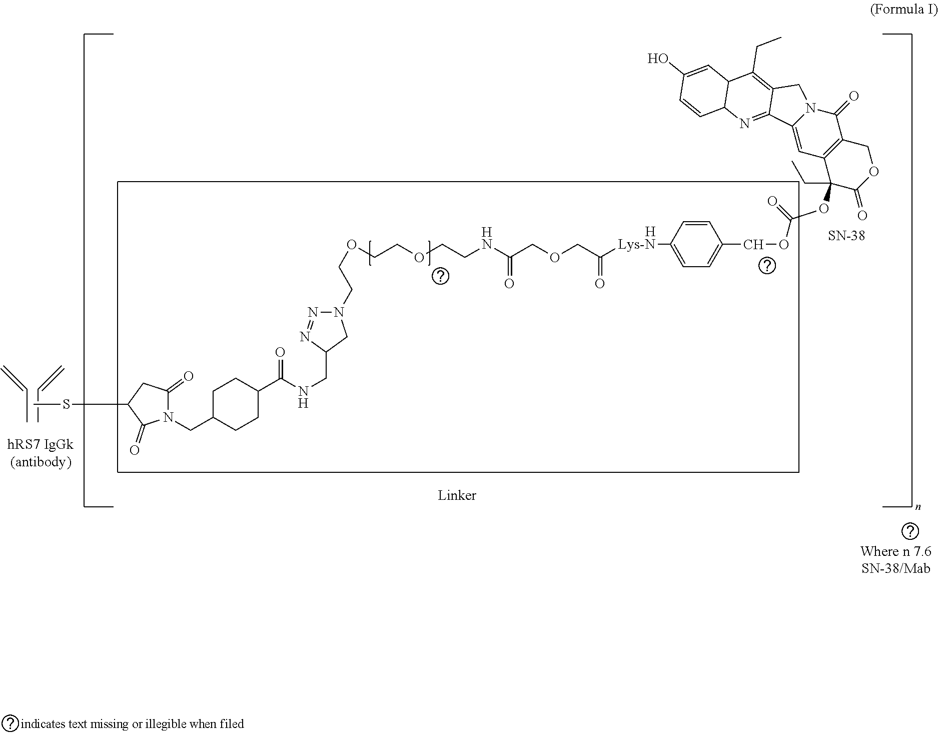

In certain embodiments, ADC comprises drug molecules linked to the antibody moieties in various stoichiometric molar ratios depending on the configuration of the antibody and, at least in part, the method used to effect configuration. In certain embodiments, the drug-antibody ratio (“DAR”) is about 7.6.

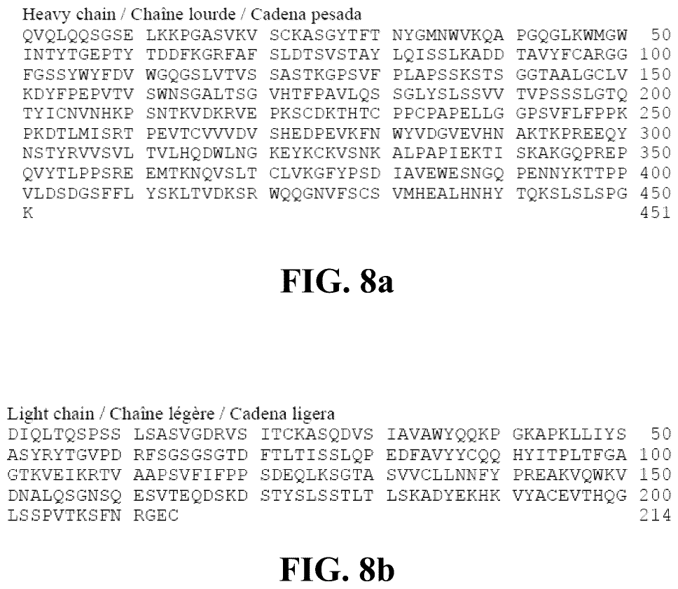

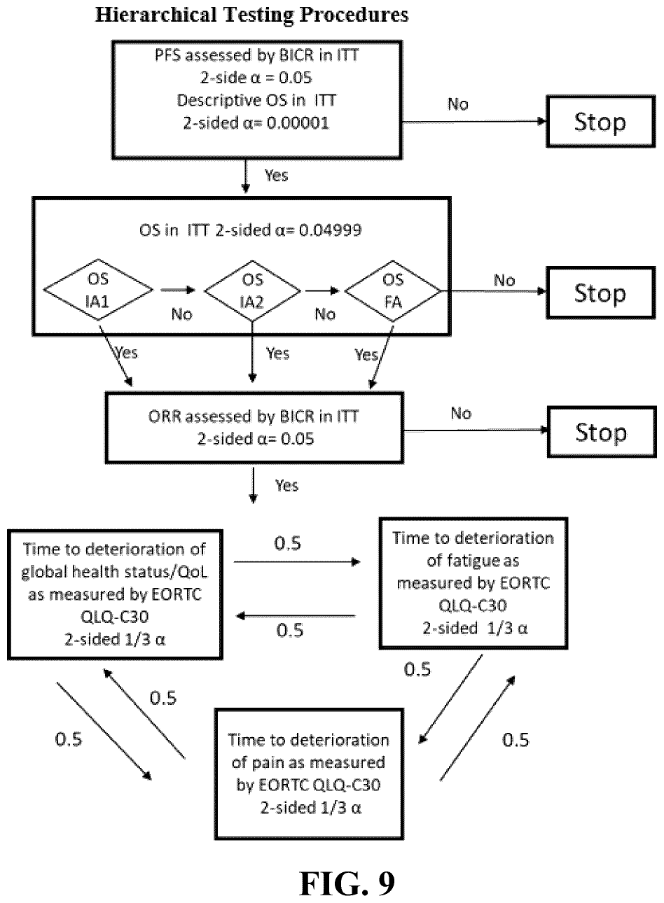

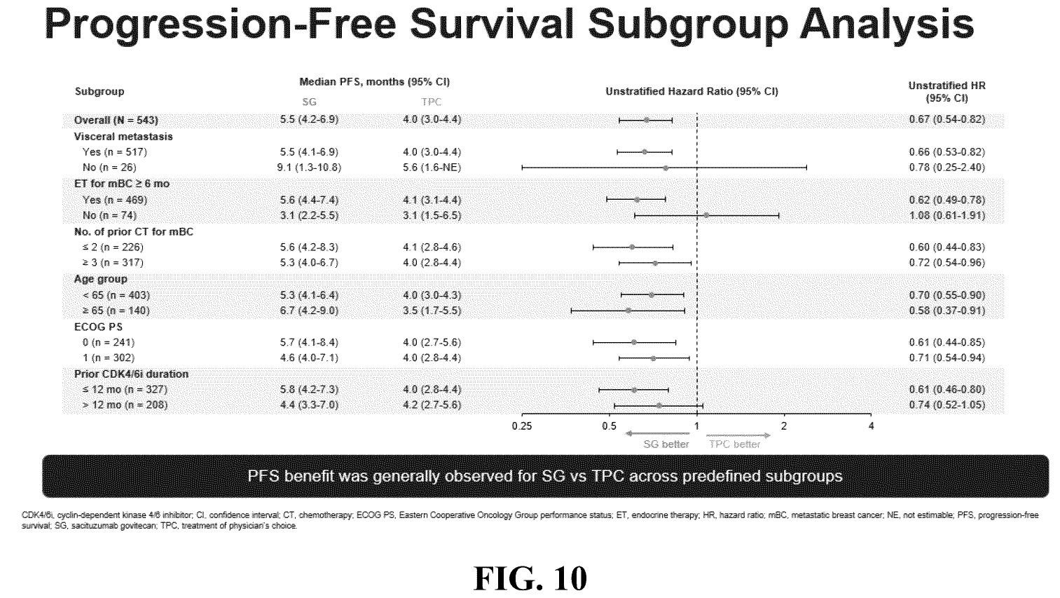

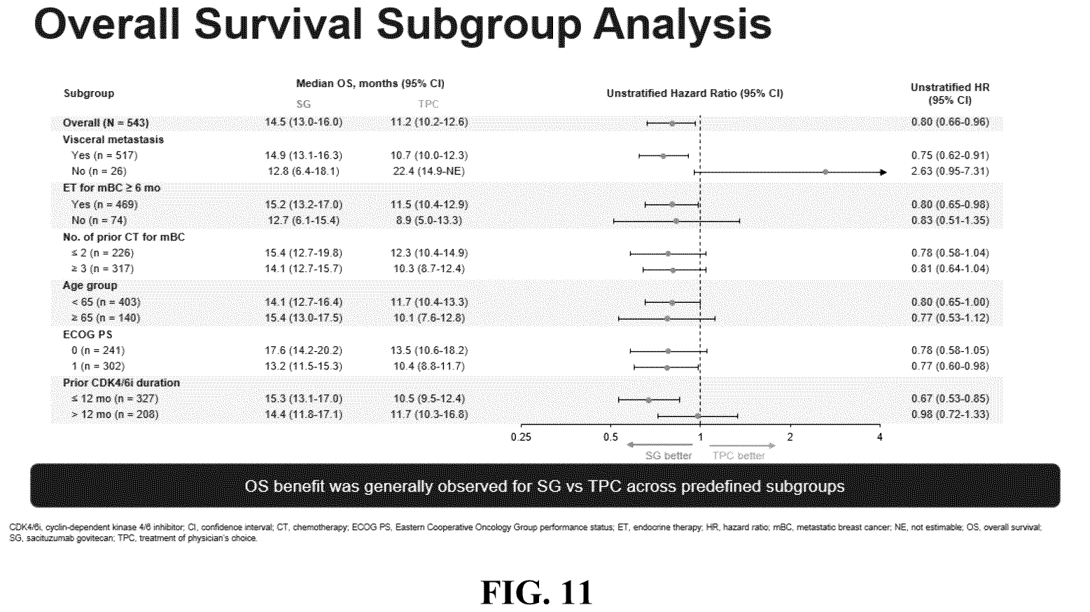

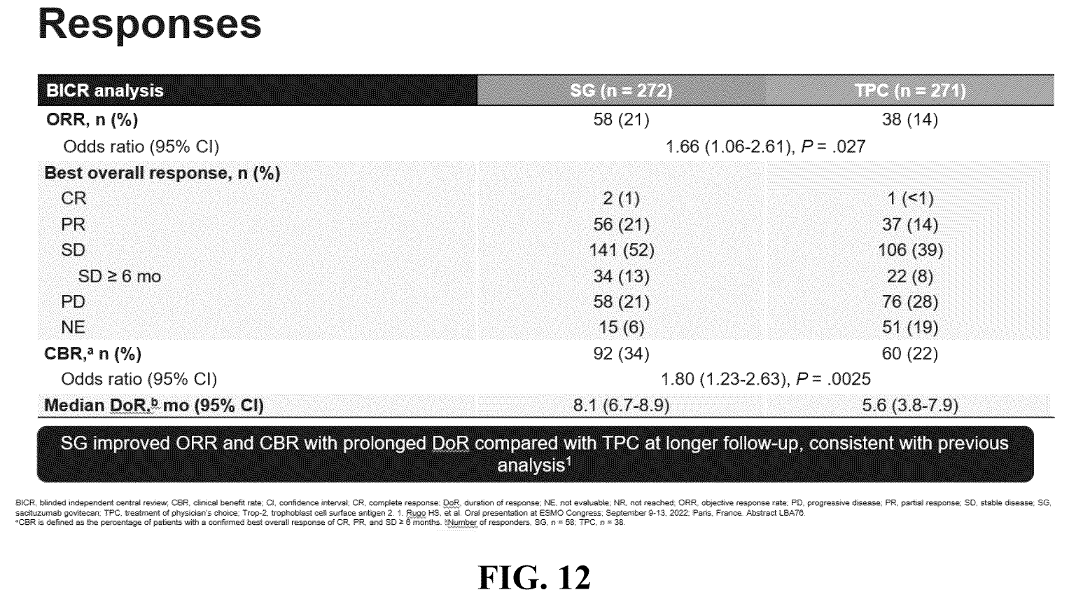

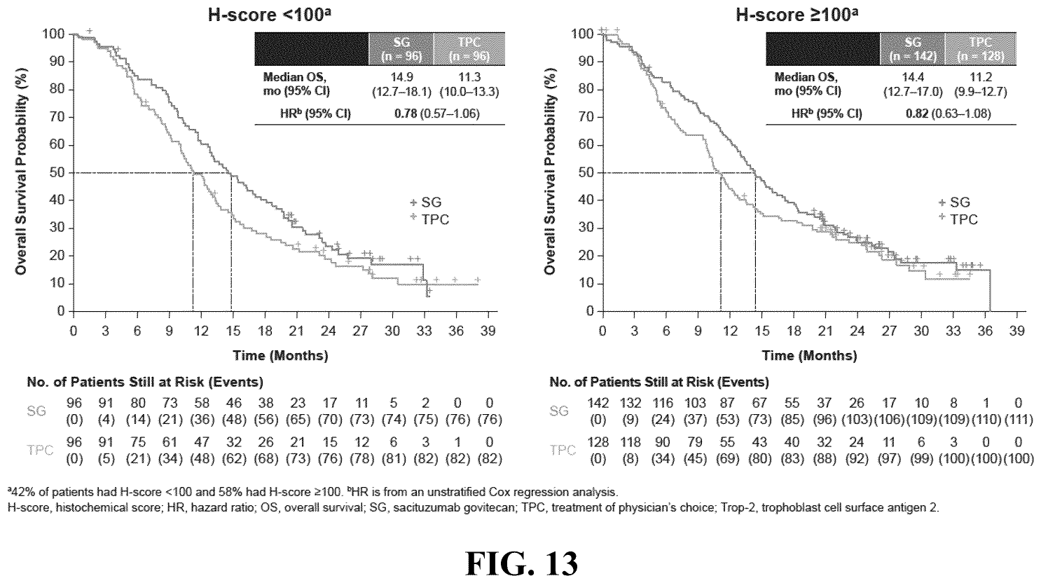

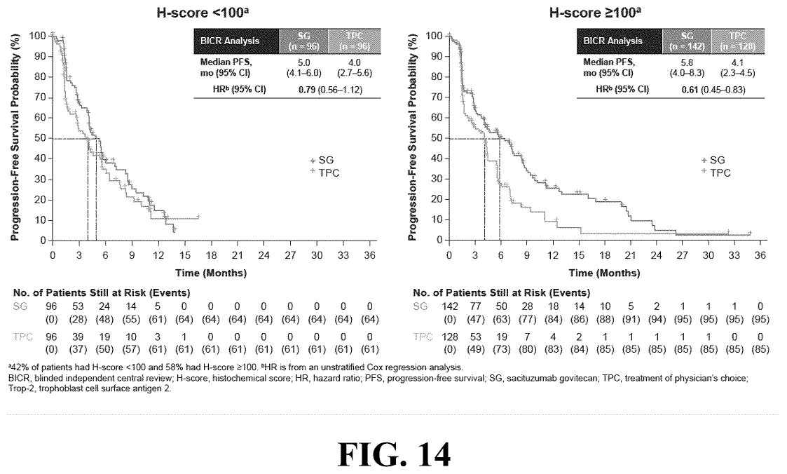

In certain embodiments, the hRS7 antibody in SG comprises the heavy chain as shown in SEQ ID NO.: 1 and light chain as shown in SEQ ID NO.: 2, and as shown in Table 9 and in FIGS. 8a-8b. In certain embodiments, the hRS7 antibody in SG comprises two heavy chains each having the sequence as shown in SEQ ID NO.: 1, and two light chains each having the sequence as shown in SEQ ID NO.: 2.

TABLE 9
Heavy chain/Chaine lourde/Cadena pesada

| | |
|---|---|
| QVQLOQSGSE LKKPGASVKV SCKASGYTFT NYGMNWVKQA PGQGLKWNGW | 50 |
| INTYTGEPTY TDDFKGRFAF SLDTSVSTAY LQISSLKADD TAVYFCARGG | 100 |
| FGSSYWYFDV WGQGSLVTVS SASTKGPSVF PLAPSSKSTS GGTAALGCLV | 150 |
| KDYFPEPVTV SWNSGALTSG VHTFPAVLQS SGLYSLSSVV TVPSSSLGTQ | 200 |
| TYICNVNHKP SNTKVDKRVE PKSCDKTHTC PPCPAPELLG GPSVFLFPPK | 250 |
| PKDTLMISRT PEVTCVVVDV SHEDPEVKFN WYVDGVEVHN AKTKPREEQY | 300 |
| NSTYRVVSVL TVLHQDWLNG KEYKCKVSNK ALPAPIEKTI SKAKGQPREP | 350 |
| QVYTLPPSRE EMTKNQVSLT CLVKGFYPSD IAVEWESNGQ PENNYKTTPP | 400 |
| VLDSDGSFFL YSKLTVDKSR WQQGNVFSCS VMHEALHNHY TQKSLSLSPG | 450 |
| K | 451 |

Light chain/Chaïne légère/Cadena ligera

| | |
|---|---|
| DIQLTQSPSS LSASVGDRVS ITCKASQDVS IAVAWYQQKP GKAPKLLIYS | 50 |
| ASYRYTGVPD RFSGSGSGTD FTLTISSLQP EDFAVYYCQQ HYITPLTFGA | 100 |
| GTKVEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC | 214 |

In certain embodiments, the hRS7 antibody in SG comprises the heavy chain as shown in SEQ ID NO.: 1 and light chain as shown in SEQ ID NO.: 2, and as shown in Table 1. In certain embodiments, the hRS7 antibody in SG comprises two heavy chains each having the sequence as shown in SEQ ID NO.: 1, and two light chains each having the sequence as shown in SEQ ID NO.: 2.

TABLE 7
Amino Acid Sequences of hRS7 antibody

| SEQ | | |
|---|---|---|
| ID NO | Description | Amino Acid Sequence |
| 1 | hRS7 heavy | QVQLQQSGSELKKPGASVKVSCKASGYTFTNYGMNWVKQAPGQGLK |
| | chain | WMGWINTYTGEPTYTDDFKGRFAFSLDTSVSTAYLQISSLKADDTA |
| | | VYFCARGGFGSSYWYFDVWGQGSLVTVSSASTKGPSVFPLAPSSKS |
| | | TSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPGK |
| 2 | hRS7 light | DIQLTQSPSSLSASVGDRVSITCKASQDVSIAVAWYQQKPGKAPKL |
| | chain | LIYSASYRYTGVPDRFSGSGSGTDFTLTISSLQPEDFAVYYCQQHY |
| | | ITPLTFGAGTKVEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGEC |
| 13 | HCDR1 | NYGMN |
| 14 | HCDR2 | WINTYTGEPTYTDDFKG |
| 15 | HCDR3 | GGFGSSYWYFDV |
| 16 | LCDR1 | KASQDVSIAVA |
| 17 | LCDR2 | SASYRYT |
| 18 | LCDR3 | QQHYITPLT |
| 19 | VH | QVQLQQSGSELKKPGASVKVSCKASGYTFTNYGMNWVKQAPGQGLK |
| | | WMGWINTYTGEPTYTDDFKGRFAFSLDTSVSTAYLQISSLKADDTA |
| | | VYFCARGGFGSSYWYFDVWGQGSLVTVSS |
| 20 | VL | DIQLTQSPSSLSASVGDRVSITCKASQDVSIAVAWYQQKPGKAPKL |
| | | LIYSASYRYTGVPDRFSGSGSGTDFTLTISSLQPEDFAVYYCQQHY |
| | | ITPLTFGAGTKVEIKR |

Exemplary anti-Trop-2 ADCs that can be used in the methods provided herein are described, for example, in U.S. Pat. Nos. 7,999,083 and 9,028,833, which are hereby incorporated herein by reference in their entireties.

### Definitions

The singular forms “a,” “an,” and “the” include the plural referents unless the context dictates otherwise.

“Antibody-drug conjugate” or “ADC” refers to a compound comprising an antibody targeting a tumor antigen and an anticancer agent drug or payload, optionally connected by a linker. In certain embodiments, the tumor antigen is tumor-associated calcium signal transducer 2 (Trop-2: NCBI Gene ID: 4070). As used herein, the tumor antigen targeted antibody is an anti-Trop-2 antibody (e.g., sacituzumab) and the drug or payload is SN38. As used herein, “ADC” refers to the compound of Formula I.

“Advanced disease” refers to stage III disease which has not spread or cancers that are unlikely to be cured or controlled long-term with treatment, as they have spread to distant locations.

“Anti-cancer agent” refers to an approved or investigational agent(s) for use, or that are being evaluated in, cancer. It can refer to systemic and targeted therapy.

“Baseline” as used herein, is the time within approximately 28 days prior to initiation of therapy, i.e., within approximately 28 days prior to day 1 of cycle 1. In certain embodiments, baseline refers to approximately day −28 to day −1 prior to day 1 of cycle 1. In certain embodiments, the baseline is the time within approximately 28 days +/−7 days prior to the initiation of therapy. In certain embodiments, baseline is the time from one measurement to another, i.e., baseline can refer to an initial time point when a measurement, i.e., a measurement of a lesion. is compared between two time points. The initial time point. in this instance, does not necessarily correspond with the initiation of treatment. In certain embodiments. “initiation of treatment refers to day 1 cycle 1. In certain embodiments. “initiation of treatment” refers to “baseline.”

“Blinded Independent Central Review” (“BICR”) refers to the process by which radiologic exams and selected clinical data, performed as part of a clinical trial protocol, are submitted to a central location for independent review.

“Clinically significant” or “clinically meaningful” (or grammatical variations thereof) refers to results/findings that improve medical care resulting in improvement in the individual's physical function his/her mental status, and ability to engage in social life. The term improvement of quality of life in medical care deals with both subjective and objective terms. Objective terms may be duration of remission of disease, etc., while subjective terms may be improvement in quality of life.

“Clinical efficacy” or “clinical activity” refers to clinical efficacy or activity in human patient(s). In certain embodiments, clinical activity or clinical efficacy refers to a partial or complete response. In certain embodiments, clinical efficacy or clinical activity, when used in the context of a population of patients, comprises ORR, DOR, and/or DCR. In certain embodiments, improvement of clinical efficacy or clinical activity, when used in the context of a comparison between two or more agents, can be used to describe a clinically meaningful benefit to the human patient of one agent compared to the other. As used herein “clinically meaningful benefit” (or grammatical variations thereof) refers to results/findings that improve medical care resulting in improvement in the individual's physical function. his/her mental status, and/or ability to engage in social life. The term improvement of quality of life in medical care deals with both subjective and objective terms. Objective terms may be duration of remission of disease, etc. Subjective terms may be improvement in the quality of life.

“Complete Response” (“CR”) refers to disappearance of all target lesions.

“Control group” as used herein, refers to a group or population of human subjects having HR+/HER2- meeting the inclusion and exclusion criteria as described herein, but randomized to receive treatment with a TPC. “Control group” also refers to a group of patients treated with the standard of care in a double arm clinical trial. i.e., a clinical trial wherein response rate is compared between a new treatment (i.e., SG) and a standard of care (i.e., “treatment of physician's choice) via randomization.

“Duration of Response” (“DOR”) is defined as the time from the date a response was first documented (CR or PR) until the date of the first documentation of disease progression or date of death (whichever occurs first). Subjects who neither progress nor die were censored on the date of their last tumor assessment. DOR was evaluated for responders (CR or PR) only.

“HR+/HER2-negative” describes a tumor that is (1) HR+ and HER2- [defined as immunohistochemistry (IHC) score of IHC 0; IHC 1+; or IHC 2+/ISH-]. “ISH” refers to in situ hybridization . “HR+/HER2-low” describes a tumor that is IHC 1+ or IHC 2+ concurrent with ISH-(e.g., lack of ERBB2 amplification). “HR+/HER2-IHC0” or “HR+/IHC0” refers to a tumor that has a IHC score of 0.

“Endocrine-based therapy,” “endocrine therapy,” or “hormone therapy” refers to one or more therapies such as tamoxifen, aromatase inhibitors (“AIs”), ovarian suppression treatment and drugs, fulvestrant, and the like, as is known in the art. In certain embodiments, “endocrine-based therapy” refers to one or more endocrine-based therapies and one or more targeted therapies, such as CDK4/CDK6 inhibitors.

“H-score.” in certain embodiments, refers to a histochemical score, which is a numerical value represented by a weighted summation of percent staining which accounts for both the staining intensity and the percentage of cells at that intensity. In certain embodiments, H-scores are calculated according to the following formula: H-score=(3 X % cells with strong intensity staining)+(2 X % cells with moderate intensity staining)+(1 X % cells with mild intensity staining). In certain embodiments, Trop 2 expression level categories are selected based on distribution of the H-score to divide the population into two groups: H-score 0<100 (Trop 2 low) and H-score≥100 (Trop 2 high).

“Intent to Treat” (“ITT) [group] is used in the context of interpretation of randomized clinical trials. Under ITT [group], study participants are analyzed as members of the treatment group to which they were randomized regardless of their adherence to, or whether they received, the intended treatment.

“Metastatic cancer or disease” is a sub-category of advanced disease. It refers to Stage IV disease and/or cancers that are unlikely to be cured or controlled long-term with treatment, as they have spread to distant locations.

“Locally advanced disease,” as used herein, refers to a disease state in which cancer cells have begun to spread out from the initial site of origin but have not yet spread to other parts of the body.

“Objective Responsive Rate” (“ORR”) refers to the proportion of subjects who have a best overall response of either complete response (CR) or partial response (PR) that is confirmed greater than or equal to 4 weeks later according to BICR using RECIST 1.1. Overall response is the best response recorded from the start of treatment until disease progression/recurrence. To be assigned a status of CR or PR, changes in tumor measurements must be confirmed by repeat assessments that should be performed greater than or equal 4 weeks later according to BICR using RECIST 1.1 after the criteria for response are first met.

“Overall Survival” (“OS”) refers to the time from the date of randomization to the date of death from any cause. Initiation of treatment [with SG or TPC] begins at the date of randomization.

“Partial Response.” (“PR”), in certain embodiments, refers to >20% increase in the sum of the LD of target lesions and a 5 mm absolute increase, taking as reference the smallest sum LD recorded since the baseline assessment or the appearance of one or more new lesions. LD is the sum of the longest diameter for all target lesions.

“Progression Free Survival” (“PFS”) refers to the time from the date of randomization to the date of the first documentation of disease progression or death (whichever occurs first) according to BICR using RECIST 1.1.

“Refractory” is used to describe when the disease does not respond to treatment or when the response to treatment does not last very long.

“Relapsed” refers to a disease that reappears or grows again after a period of remission.

“Severe neutropenia” as used herein, is defined as grade 4 neutropenia for 7 or more days. grade 3-4 febrile neutropenia, or, at the time of scheduled treatment, grade 3-4 neutropenia which delays dosing by 2 or 3 weeks for recovery to grade 1 or less as shown in Table 1a and Table 1b.

“Standard of care,” as used herein refers to a preferred treatment regimen for a particular disease or indication, such as a treatment that a governmental regulatory agency has approved for the disease or indication.

“Statistically significant” or “statistically significantly superior” (and grammatical variations thereof) refers, in certain embodiments, to meeting primary and secondary endpoints of the clinical trial. In certain embodiments, the term refers to whether there is any mathematical significance to the carried analysis of the results or not.

“Targeted therapie(s)” refers to small molecule inhibitors, monoclonal antibodies, tumor agnostic treatments, CDK4/CDK6 inhibitors (e.g., palbociclib, ribociclib, abemaciclib), everolimus. alpelisib) in cancer treatment.

“Taxane(s)” is a type of chemotherapy as is known in the art.

“Therapeutically effective amount” or “effective amount” is defined as an amount of compound/drug to treat the disease or disorders and achieve clinical efficacy.

“Treatment” (or grammatical variations thereof), refers to medical care that results in clinically meaningful benefit or clinically meaningful improvement. In certain embodiments, “treatment” refers to medical care that results in a clinically meaningful improvement in OS compared to a standard-of-care, such as a TPC as used herein. In certain embodiments, “treatment” refers to medical care that results in a clinically meaningful improvement in OS and/or PFS.

“Time to Deterioration” (“TTD”) is defined as the time between randomization and the time a subject experienced a deterioration (i.e., ≥10 points worsening from baseline in a given domain).

“Treatment of Physician's Choice” (“TPC”), as used herein, is a single-agent treatment determined before randomization to one of the following: (1) Eribulin (1.4 mg/m² for North American sites, 1.23 mg/m² for EU sites or per institution) administered as an IV on days 1 and 8 of one or more 21-day cycles; (2) Capecitabine (1000-1250 mg/m²) administered PO twice daily for 2 weeks followed by a 1-week rest period given as a 21-day cycle; (3) Gemcitabine (800-1200 mg/m²) administered via IV on days 1, 8, and 15 of each 28-day cycle or per institution; or (4) Vinorelbine (25 mg/m² IV on day 1 weekly cycle per institution). (Note: subjects with grade 2 neuropathy are eligible. but should not receive vinorelbine as TPC). No combination of the (4) choices was permitted. In certain embodiments, “TPC” is the standard-of-care.

### Recommended Dosing Regimen of SG

In certain embodiments, SG is administered once weekly on days 1 and 8 of continuous 21-day treatment cycles. In certain embodiments, SG is administered at a dosage of 10 mg/kg of SG once weekly on days 1 and 8 of continuous 21-day treatment cycles. In certain embodiments, SG is administered until disease progression or unacceptable toxicity. In certain embodiments, the dosage may be reduced, i.e., by 25% or by 50%, due to adverse reactions, but still be administered once weekly on days 1 and 8 of continuous 21-day treatment cycles.

In certain embodiments, disclosed herein are methods of treating HR+/HER2- breast cancer comprising administering, to a patient in need thereof, SG as an intravenous infusion once weekly on day 1 and day 8 of one or more 21-day treatment cycles. In certain embodiments, disclosed herein are methods of treating HR+/HER2- breast cancer comprising administering, to a patient in need thereof, 10 mg/kg SG as an intravenous infusion once weekly on day 1 and day 8 of one or more 21-day treatment cycles. In certain embodiments, the method comprises administering SG as an intravenous infusion once weekly on day 1 and day 8 of at least (2); (3); (4); (5); (6); (7); (8); (9); (10); (11); (12); (13); (14); (15); (16); (17); (18); (19) or (20) 21-day treatment cycles. In certain embodiments, the method comprises administering 10 mg/kg SG as an intravenous infusion once weekly on day 1 and day 8 of at least (2); (3); (4); (5); (6); (7); (8); (9); (10); (11); (12); (13); (14); (15); (16); (17); (18); (19) or (20) 21-day treatment cycles.

In certain embodiments, disclosed herein are methods of achieving an overall survival of at least about 14 months from the initiation of treatment with SG and/or methods of increasing overall survival in a patient having HR+/HER2- breast cancer, comprising administering SG as an intravenous infusion once weekly on day 1 and day 8 of at least (2); (3); (4); (5); (6); (7); (8); (9); (10); (11); (12); (13); (14); (15); (16); (17); (18); (19) or (20) 21-day treatment cycles. In certain embodiments, the method comprises administering 10 mg/kg SG as an intravenous infusion once weekly on day 1 and day 8 of at least (2); (3); (4); (5); (6); (7); (8); (9); (10); (11); (12); (13); (14); (15); (16); (17); (18); (19) or (20) 21-day treatment cycles.

In certain embodiments, disclosed herein are methods of achieving an overall survival of at least about 14 months from the initiation of treatment with SG and/or methods of increasing overall survival in a patient having HR+/HER2- breast cancer, comprising administering 10 mg/kg SG as an intravenous infusion once weekly on day 1 and day 8 of at least (2); (3); (4); (5); (6); (7); (8); (9); (10); (11); (12); (13); (14); (15); (16); (17); (18); (19) or (20) 21-day treatment cycles. In certain embodiments. the method comprises administering 10 mg/kg SG as an intravenous infusion once weekly on day 1 and day 8 of at least (2); (3); (4); (5); (6); (7); (8); (9); (10); (11); (12); (13); (14); (15); (16); (17); (18); (19) or (20) 21-day treatment cycles.

In certain embodiments, 10 mg/kg SG is administered as intravenous infusion once weekly on day 1 and day 8 of one or more 21-day treatment cycles. In certain embodiments, the patient achieves a PFS of at least about 5.5 months. As disclosed herein, patients receive SG until disease progression, unacceptable toxicity, or death. For example, patients that have PFS for at least about 5.5 months have either a complete response or a partial response for at least about 5.5 months and thus are being treated with SG for at least about 5.5 months, i.e., being treated with at least about 8 cycles of SG.

In certain embodiments, 10 mg/kg SG is administered as an intravenous infusion once weekly on day 1 and day 8 of one or more 21-day treatment cycles. In certain embodiments, the patient has a duration of response (DOR) of at least about 8 months. Once PR or CR is reached, DOR is the amount of time until PD or death. At PD or death, treatment is discontinued. Thus, for example, a patient that has a duration of response of at least about 8 months is being treated for at least about 8 months, i.e., at least about (11) 21-day cycles.

In certain embodiments, 10 mg/kg SG is administered as an intravenous infusion once weekly on day 1 and day 8 of one or more treatment cycles. In certain embodiments, the patient achieves an overall survival of at least about 14 months. In certain embodiments, a patient with an overall survival of at least about 14 months is being treated with SG during that time, and, as such. is being treated with at least about (19) 21-day cycles of SG.

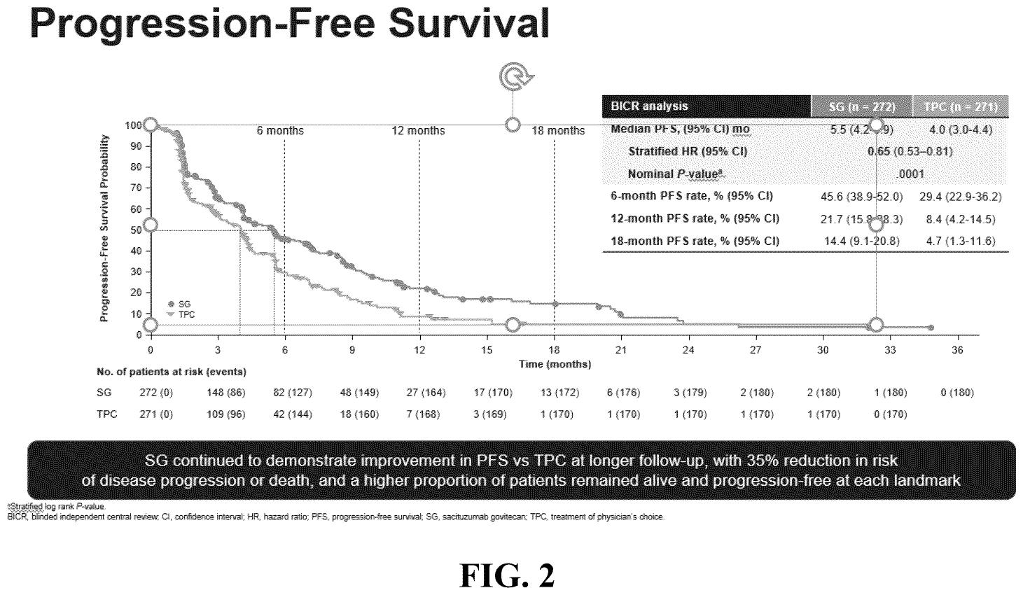

In certain embodiments, and as shown in FIG. 2, the patient achieves a PFS of at least about 18 months. PFS refers to the date of randomization to the date of first documentation of disease progression or death (whichever occurs first) according to RECIST 1.1. As such, the patient is treated with SG for at least about 18 months, or at least about (19) 21-day treatment cycles.

In certain embodiments, the patient is treated with at least about (8) 21-day cycles of SG; at least about (9) 21-day cycles of SG; at least about (10) 21-day cycles of SG; at least about (11) 21-day cycles of SG; at least about (12) 21-day cycles of SG; at least about (13) 21-day cycles of SG; at least about (14) 21-day cycles of SG; at least about (15) 21-day cycles of SG; at least about (16) 21-day cycles of SG; at least about (17) 21-day cycles of SG; at least about (18) 21-day cycles of SG; at least about (19) 21-day cycles of SG; at least about (20) 21-day cycles of SG; at least about (30) 21-day cycles of SG; or at least about (40) 21-day cycles of SG.

In certain embodiments, and as shown, for example, in FIGS. 2-3, the patient is treated with the methods disclosed herein for at least about 12 months, at least about 13 months, at least about 14 months, at least about 15 months, at least about 16 months, at least about 17 months, or at least about 18 months.

### Dose Modifications for Adverse Reactions

In certain embodiments, the dosage of 10 mg/kg of SG is withheld, modified, or discontinued to manage adverse reactions as described in Tables 1a and 1b. In certain embodiments, the dosage of SG is not re-escalated after a dose reduction for an adverse reaction has been made.

TABLE 1a
Dose Modifications for Adverse Reactions (Severe Neutropenia)

| | | |
|---|---|---|
| Adverse Reaction | Occurrence | Dose Modification |

Severe Neutropenia [see Warnings and Precautions (5.1)]

| | | |
|---|---|---|
| Grade 4 neutropenia ≥7 days | First | 25% dose reduction and administer |
| OR | | granulocyte-colony stimulating |
| Grade 3-4 febrile neutropenia, | | factor (G-CSF). |
| OR | Second | 50% dose reduction and administer |
| At time of scheduled treatment, Grade 3-4 neutropenia which | | G-CSF. |
| delays dosing by 2 or 3 weeks for recovery to ≤Grade 1 | Third | Discontinue treatment and |
| | | administer G-CSF. |
| At time of scheduled treatment, Grade 3-4 neutropenia which | First | Discontinue treatment and |
| delays dosing beyond 3 weeks for recovery to ≤Grade 1 | | administer G-CSF. |

TABLE 1b
Dose Modifications for Adverse Reactions (Severe Non-Neutropenic Toxicity)

| | | |
|---|---|---|
| Adverse Reaction | Occurrence | Dose Modification |

Severe Non-Neutropenic Toxicity

| | | |
|---|---|---|
| Grade 4 non-hematologic toxicity of any duration, | First | 25% dose reduction |
| OR | Second | 50% dose reduction |
| Any Grade 3-4 nausea, vomiting or diarrhea due to treatment that | Third | Discontinue treatment |
| is not controlled with antiemetics and anti-diarrheal agents [see | | |
| Warnings and Precautions (5.2, 5.4)], | | |
| OR | | |
| Other Grade 3-4 non-hematologic toxicity persisting >48 hours | | |
| despite optimal medical management, | | |
| OR | | |
| At time of scheduled treatment, Grade 3-4 non-neutropenic | | |
| hematologic or non-hematologic toxicity, which delays dose by 2 | | |
| or 3 weeks for recovery to ≤Grade 1 | | |
| In the event of Grade 3-4 non-neutropenic hematologic or non- | First | Discontinue treatment |
| hematologic toxicity, which does not recover to ≤Grade 1 within | | |
| 3 weeks | | |

### Treatment Prior to Administration of SG (or TPC)

In certain embodiments, methods of treating a HR+/HER2-breast cancer in a human subject are disclosed herein. In certain embodiments, the HR+/HER2- breast cancer is a HR+/HER2-low breast cancer. In certain embodiments, the HR+/HER2-breast cancer is a HR+/HER2-IHC0 breast cancer. In certain embodiments, the HR+/HER2- breast cancer is an unresectable, locally advanced breast cancer. In certain embodiments, the HR+/HER2- breast cancer is metastatic breast cancer. In certain embodiments, the HR+/HER2-breast cancer is relapsed. In certain embodiments, the HR+/HER2- breast cancer is refractory. In certain embodiments, the HR+/HER2- breast cancer is relapsed and refractory. In certain embodiments, the HR+/HER2- breast cancer has relapsed after endocrine-based therapy. In certain embodiments, the HR+/HER2- breast cancer is refractory to endocrine-based therapy.

In certain embodiments, the HR+/HER2- breast cancer is refractory to CDK4/CDK6 inhibitor therapy. In certain embodiments, the HR+/HER2- breast cancer is relapsed after CDK4/CDK6 inhibitor therapy. In certain embodiments, the HR+/HER2- breast cancer is relapsed and refractory to CDK4/CDK6 inhibitor therapy.

In certain embodiments, the HR+/HER2- breast cancer is relapsed and/or refractory to taxane therapy. In certain embodiments, the HR+/HER2- breast cancer has relapsed after taxane therapy. In certain embodiments, the HR+/HER2- breast cancer is refractory to taxane therapy. In certain embodiments, the HR+/HER2- breast cancer is relapsed and/or refractory to CDK4/CDK6 inhibitor therapy, to endocrine therapy, and to taxane therapy. In certain embodiments, the HR+/HER2- breast cancer has relapsed after CDK4/CDK6 inhibitor therapy, endocrine therapy, and taxane therapy. In certain embodiments, the HR+/HER2- breast cancer is refractory to CDK4/CDK6 inhibitor therapy. endocrine therapy, and taxane therapy.

In certain embodiments, the HR+/HER2-low breast cancer is refractory to CDK4/CDK6 inhibitor therapy. In certain embodiments, the HR+/HER2- low breast cancer has relapsed after CDK4/CDK6 inhibitor therapy. In certain embodiments, the HR+/HER2-low breast cancer is relapsed or refractory to CDK4/CDK6 inhibitor therapy, endocrine therapy, and taxane therapy.

In certain embodiments, the HR+/HER2-IHC0 breast cancer is refractory to CDK4/CDK6 inhibitor therapy. In certain embodiments, the HR+/HER2- IHC0 breast cancer is relapsed after CDK4/CDK6 inhibitor therapy. In certain embodiments, the HR+/HER2- IHC0 breast cancer is refractory or refractory to CDK4/CDK6 inhibitor therapy, endocrine therapy, and taxane therapy.

In certain embodiments, prior to treatment of the HR+/HER2- breast cancer in a human subject with the methods disclosed herein, the subject has had at least two prior chemotherapies, but no more than four prior chemotherapies. In certain embodiments, prior to treatment of the HR+/HER2- breast cancer with the methods disclosed herein, the human subject has had at least three prior therapies. In certain embodiments, adjuvant or neoadjuvant therapy for early-stage disease qualifies as one of the required chemotherapy regimens if the development of unresectable, locally advanced, or metastatic disease occurred within a 12-month period of time of adjuvant and neoadjuvant therapy. In certain embodiments, treatment for bone metastases or hormonal therapy is not considered as one of the required chemotherapy regimens. In certain embodiments, prior to the treatment of the HR+/HER2- breast cancer with the methods disclosed herein, the human subject has had at least two, but no more than four, prior chemotherapies, at least one of which was in the metastatic setting. In certain embodiments, prior to the treatment of the HR+/HER2- breast cancer with the methods disclosed herein, the human subject has had at least two chemotherapies in the metastatic setting.

In certain embodiments, prior to treatment of the HR+/HER2- breast cancer with the methods disclosed herein, the human subject has had prior chemotherapy in the (neo)adjuvant setting. In certain embodiments, prior to treatment of the HR+/HER2- breast cancer with the methods disclosed herein, the human subject has had ≥6 months of prior endocrine therapy in the metastatic setting. In certain embodiments, prior to treatment of the HR+/HER2- breast cancer with the methods disclosed herein, the human subject has had prior CDK4/CDK6 inhibitor therapy for ≤12 months. In certain embodiments, prior to treatment of the HR+/HER2- breast cancer with the methods disclosed herein, the human subject has had prior CDK4/CDK6 inhibitor therapy for >12 months. In certain embodiments, prior to treatment of the HR+/HER2- breast cancer with the methods disclosed herein, the human subject has had ≤2 prior lines of chemotherapy. In certain embodiments, prior to treatment of the HR+/HER2- breast cancer with the methods disclosed herein, the human subject has had ≥3 prior lines of chemotherapy. In certain embodiments, prior to treatment of the HR+/HER2- breast cancer with the methods disclosed herein, the human subject has had visceral metastases at baseline. In certain embodiments, prior to treatment of the HR+/HER2- breast cancer with the methods disclosed herein, the human subject has had liver metastases. In certain embodiments, prior to treatment of the HR+/HER2- breast cancer with the methods disclosed herein, the human subject has had de novo metastatic breast cancer. In certain embodiments, prior to treatment of the HR+/HER2- breast cancer with the methods disclosed herein, the human subject has an ECOG PS (Eastern Cooperative Oncology Group Performance Status) of 0. In certain embodiments, prior to the treatment of the HR+/HER2- breast cancer with the methods disclosed herein, the human subject has an ECOG of 1. In certain embodiments, the human subject is female. In certain embodiments, prior to the treatment of the HR+/HER2- breast cancer with the methods disclosed herein, the human subject is <65 years of age. In certain embodiments, prior to the treatment of the HR+/HER2- breast cancer with the methods disclosed herein, the human subject is ≥65 years of age.

In certain embodiments, prior to treatment of the HR+/HER2- breast cancer in the human subject with the methods disclosed herein, the subject has had CDK4/CDK6 inhibitor therapy in any setting. In certain embodiments, prior to treatment of the HR+/HER2- breast cancer in the human subject with the methods disclosed herein, the subject has had CDK4/CDK6 inhibitor therapy in the metastatic setting. In certain embodiments, prior to treatment of the HR+/HER2- breast cancer in the human subject with the methods disclosed herein, the subject has had endocrine-based therapy in any setting. In certain embodiments, prior to treatment of the HR+/HER2- breast cancer in the human subject with the methods disclosed herein, the subject has had endocrine-based therapy in the metastatic setting. In certain embodiments, prior to treatment of the HR+/HER2- breast cancer in the human subject with the methods disclosed herein, the subject has had endocrine-based therapy and at least two additional therapies in the metastatic setting. In certain embodiments, prior to treatment of the HR+/HER2- breast cancer in the human subject with the methods disclosed herein, the subject has had endocrine-based therapy and at least two additional therapies in any setting. In certain embodiments, prior to treatment of the HR+/HER2- breast cancer in the human subject using the methods disclosed herein, the subject has had at least one endocrine-based therapy comprising CDK4/CDK6 inhibitor therapy and at least one taxane therapy. In certain embodiments, the taxane is selected from the group consisting of docetaxel and paclitaxel. In certain embodiments, prior to treatment of the HR+/HER2- breast cancer in the human subject with the methods disclosed herein. the subject has had at least one endocrine-based therapy comprising CDK4/CDK6 inhibitor therapy and least two additional systemic therapies. In certain embodiments, prior to the treatment of the HR+/HER2- breast cancer with the methods as disclosed herein, the subject has had at least one endocrine-based therapy comprising a CDK4/CDK6 inhibitor therapy, and at least two additional systemic therapies, one of which was a taxane.

In certain embodiments, prior to treatment of the HR+/HER2- breast cancer in the human subject with the methods disclosed herein. the subject has not had previous treatment with a topoisomerase I inhibitor as a free form or as other formulations. In certain embodiments, prior to treatment of the HR+/HER2- breast cancer in the human subject with the methods disclosed herein. the subject has not had treatment with an antibody-drug conjugate. In certain embodiments, prior to treatment of the HR+/HER2- breast cancer with the methods disclosed herein, the subject has not had treatment with an antibody drug conjugate, wherein the drug in the ADC is a topoisomerase I inhibitor.

In certain embodiments, the CDK4/CDK6 inhibitor therapy is selected from the group consisting of palbociclib, ribociclib. and abemaciclib. In certain embodiments, the CDK4/CDK6 inhibitor therapy is palbociclib. In certain embodiments, the CDK4/CDK6 inhibitor therapy is ribociclib. In certain embodiments, the CDK4/CDK6 inhibitor therapy is abemaciclib. In certain embodiments, the CDK4/CDK6 inhibitor therapy is one or more of palbociclib, ribociclib, and abemaciclib. In certain embodiments, the CDK4/CDK6 inhibitor therapy is selected from those known in the art.

### Efficacy

Overall Survival—In certain embodiments, treatment of a HR+/HER2- breast cancer in a human subject with a dosage of 10 mg/kg of SG achieves an OS (from initiation of treatment with SG) of at least 12 months, at least 13 months, at least 14 months, at least 14.4 months, at least 14.5 months, at least 15 months, at least 16 months, at least 17 months, at least 18 months, at least 19 months, or at least 20 months. In certain embodiments, treatment of a HR+/HER2- breast cancer in a human subject with a dosage of 10 mg/kg of SG achieves an OS of at least 14 months from initiation of treatment with SG. In certain embodiments, treatment of a HR+/HER2- breast cancer in a human subject with a dosage of 10 mg/kg of SG achieves an OS of at least 14.4 months from initiation of treatment with SG. In certain embodiments, treatment of a HR+/HER2- breast cancer in a human subject with 10 mg/kg of SG achieves an OS of at least 14.5 months from initiation of treatment with SG. In certain embodiments, treatment of a HR+/HER2- breast cancer in a human subject with 10 mg/kg of SG achieves an OS of 14.4 from initiation of treatment with SG. In certain embodiments, treatment of a HR+/HER2- breast cancer in a human subject with 10 mg/kg of SG achieves an OS of 14.5 from initiation of treatment with SG.

In certain embodiments, treatment of a HR+/HER2- breast cancer in a population of human subjects with SG achieves a median OS of 14.5 months and treatment of HR+/HER2- breast cancer in a control group of human subjects with TPC achieves a median OS of 11.2 months.

In certain embodiments. and as shown in Table 2 and FIG. 3. treatment of HR+/HER2- breast cancer in a population of human subjects with SG achieves a median OS of 14.5 months and treatment of a HR+/HER2- breast cancer in a control group of human subjects with TPC achieves a median OS of 11.2 months at a median time point of 12.75 months (confidence interval. or “CI” 95%). Table 2 shows results from a data cutoff date of Dec. 1, 2022. At this data cutoff, 437 OS events had occurred. with 47 new deaths since 2^{nd} planned interim analysis.

In certain embodiments, treatment of HR+/HER2- breast cancer in a population of human subjects with SG achieves a median OS (from initiation of treatment with SG) of 14.5 months, 14.6 months, 14.7 months, 14.8 months, 14.9 months, 15 months, 16 months, 17 months, 18 months, 19 months and 20 months; and treatment of HR+/HER2- breast cancer in a control group of human subjects with TPC achieves a median OS of 1 months, 2 months, 3 months, 4 months, 5 months, 6 months, 7 months, 8 months, 9 months, 10 months, 11 months, 11.2 months, or 12 months.

In certain embodiments, treatment of HR+/HER2- breast cancer in a population of human subjects with SG achieves a 12-month OS rate of 60.9% and treatment of a HR+/HER2- breast cancer in a control group of human subjects with TPC achieves a 12-month OS rate of 47.1% (95% CI). In certain embodiments, treatment of HR+/HER2-breast cancer in a population of human subjects with SG achieves an 18-month OS of 39.2% and treatment of HR+/HER2- breast cancer in a control group of human subjects with TPC achieves an 18-month OS of 31.7% (95% CI). In certain embodiments, treatment of a HR+/HER2- breast cancer in a population of human subjects with SG achieves a 24-month OS rate of 25.7% and treatment of HR+/HER2- breast cancer in a control group of human subjects with TPC achieves a 24-month OS rate of 21.2% (95% CI).

In certain embodiments, treatment of a HR+/HER2- breast cancer in a population of human subjects with SG achieves a 12-month OS rate that is statistically significantly greater than the 12-month OS rate of a control group of human subjects treated with TPC. In certain embodiments, treatment of a HR+/HER2- breast cancer in a population of human subjects with SG achieves an 18-month OS rate that is statistically significantly greater than the 18-month OS rate of a control group of human subjects treated with TPC. In certain embodiments, treatment of HR+/HER2- breast cancer in a population of human subjects with SG achieves a 24-month OS rate that is statistically significantly greater than the 24-month OS rate of a control group of human subjects treated with TPC.

In certain embodiments, treatment of HR+/HER2- breast cancer in a population of human subjects with SG achieves a median OS that is statistically significantly greater than treatment of HR+/HER2- breast cancer in a control group of human subjects with TPC.

TABLE 2
Final OS Results from TROPiCS-02 Clinical Trial -
Data Cutoff - Dec. 1, 2022 (about 437 OS results had occurred)

| | |
|---|---|
| SG (n = 272) | TPC (n = 271) |

| | |
|---|---|
| Median OS, mo (95% CI) | 14.5 (13.0-16.0) 11.2 (10.2-12.6) |
| Stratified HR (95% CI) | 0.79 (0.65-0.95) |
| Nominal P-value^{a} | 0.0133 |
| 12-month OS rate, % (95% CI) | 60.9 (54.8-66.4) 47.1 (41.0-63.0) |
| 18-month OS rate, % (95% CI) | 39.2 (33.4-45.0) 31.7 (26.2-37.4) |
| 24-month OS rate, % (95% CI) | 25.7 (20.5-31.2) 21.1 (16.3-26.3) |

Progression-Free Survival—In certain embodiments, treatment of HR+/HER2- breast cancer in a population of human subjects with SG achieves a median PFS (from initiation of treatment with SG) of at least 4 months, at least 5 months, at least 6 months, at least 7 months, at least 8 months, at least 9 months, and/or at least 10 months. In certain embodiments, treatment of HR+/HER2- breast cancer in a population of human subjects with SG achieves a median PFS of 5.5 months from initiation of treatment with SG.

In certain embodiments, and as shown in Table 3 (Dec. 1, 2022 data cut-off) and FIG. 2, treatment of HR+/HER2- breast cancer in a population of human subjects with SG achieves a median PFS of 5.5 months, and treatment of HR+/HER2- breast cancer in a control group of human subjects achieves a median PFS of 4.0 months [Stratified HR (95% CI) 0.65 (0.53-0.81), Nominal P-value 0.0001]. In certain embodiments, treatment of HR+/HER2-breast cancer in a population of human subjects with SG achieves a median PFS of about 5 months, 5.5 months, 6 months, 7 months, 8 months, 9, months, and 10 months: and treatment of HR+/HER2- breast cancer in a control group of human subjects with TPC achieves a median PFS of about 1 months, 2 months, 3 months, 4 months, 5 months, and 6 months.

In certain embodiments, and as shown in Table 3, treatment of HR+/HER2- breast cancer in a population of human subjects with SG achieves a 6-month PFS rate of 45.6% and treatment of HR+/HER2 in a control group with TPC achieves a 6-month PFS of 29.4% (95% CI). In certain embodiments, treatment of HR+/HER2- breast cancer in a population of human subjects with SG achieves a 12-month PFS of 21.7% and treatment of HR+/HER2- breast cancer in a control group with TPC achieves a 12-month PFS of 8.4% (95% CI). In certain embodiments, treatment of HR+/HER2- breast cancer in a population of human subjects with SG achieves an 18-month PFS of 14.4% and treatment of HR+/HER2- breast cancer in a control group of human subjects with TPC achieves an 18-month PFS of 4.7% (95% CI).

TABLE 3
Final PFS Results from the TROPiCS-02 Clinical
Trial (Data Cut-Off - Dec. 2, 2022)

| BICR analysis | SG (n = 272) | TPC (n = 271) |
|---|---|---|
| Median PFS, (95% CI) mo | 5.5 (4.2-6.9) | 4.0 (3.0-4.4) |
| Stratified HR (95% CI) | 0.65 (0.53-0.81) | |
| Nominal P-value^{a} | .0001 | |
| 6-month PFS | 45.6 (38.9-52.0) | 29.4 (22.9-36.2) |
| rate, % (95% CI) | | |
| 12-month PFS | 21.7 (15.8-28.3) | 8.4 (4.2-14.5) |
| rate, % (95% CI) | | |
| 18-month PFS | 14.4 (9.1-20.8) | 4.7 (1.3-11.6) |
| rate, % (95% CI) | | |

Duration of Response—DOR: In certain embodiments, and as shown in Table 4, treatment of HR+/HER2- breast cancer in a population of human subjects with SG achieves a median duration of response of about 6 months, of about 7 months, of about 8 months, of about 9 months, of about 10 months, of about 11 months, or of about 12 months. In certain embodiments, treatment of HR+/HER2- breast cancer in a population of human subjects with SG achieves a median duration of response of 8.1 months.

In certain embodiments, treatment of HR+/HER2- breast cancer in a population of human subjects with SG achieves a median DOR of 8.1 months and treatment of HR+/HER2- breast cancer in a control group of human subjects with TPC achieves a median DOR of 5.6 months (95% CI).

TABLE 4
Summary of Efficacy Results from TROPiCS-02 Clinical Trial

| Patients, ITT² | SG (n = 272) | TPC (n = 271) |
|---|---|---|
| Median PFS,^{a} mo | 5.5 (4.2-7.0) | 4.0 (3.1-4.4) |
| HR (95% CI), P-value | 0.66 (0.53-0.81), P = .0003 | |
| Median OS, mo | 14.4 (13.0-15.7) | 11.2 (10.1-12.7) |
| HR (95% CI), P-value | 0.79 (0.65-0.96), P = .020 | |
| ORR,^{a} % | 21% | 14% |
| Odds ratio | 1.63 (1.03-2.56), P = .035 | |
| (95% CI), P-value | | |
| Median DoR,^{a} | 8.1 (6.7-8.9) | 5.6 (3.8-7.9) |
| mo (95% CI) | | |

In certain embodiments, the human subject treated SG as disclosed herein is homozygous for the UGT1A1*28 allele. In certain embodiments, the human subject treated with SG as disclosed herein is heterozygous for the UGT1A1*28 allele. In certain embodiments, the human subject treated with SG as disclosed herein is homozygous for the UGT1A1*28 allele or heterozygous for the UGT1A1*28 allele. In certain embodiments, the dosage of 10 mg/kg of SG is not modified or reduced based on UGTIA1*28 status.

### EXAMPLES

### Example 1

### Phase 1/2 Clinical Trial—IMMU-132-01 (NCT01631552)

SG was evaluated in patients with previously treated HR+HER2- metastatic breast cancer in a single-arm, basket trial, as shown in Tables 5 and 6. 54 women with HR+/HER2- breast cancer were enrolled. All received at least two prior lines of therapy (in any setting), of which one must have been endocrine-based therapy. As shown in Table 5, all 54 patients (100%) previously received endocrine therapy; 46 patients (85.2%) previously received taxane, 36 patients (66.7%) previously received anthracycline, 35 patients (64.8%) previously received capecitabine, 33 (61%) previously received a CDK 4/6 inhibitor, and 24 patients (44.4%) previously received an mTOR inhibitor. The inclusion criteria for this basket trial, in terms of prior lines of therapy, and types, was broader than the inclusion criteria for TROPiCS-02. The inclusion criteria for TROPiCS-02 required more lines of therapy and required previous treatment with a CDK4/CDK6 inhibitor.

TABLE 5
Patient Characteristics from the IMMU-132-01 Study

| | |
|---|---|
| ≥1 prior chemotherapy for metastatic disease | 96 |
| ≥2 prior chemotherapy regimens for metastatic disease | 76 |
| Median number of metastatic chemotherapy lines (range) | 2 (0-11) |
| Prior chemotherapy for metastatic disease, % | |
| Taxanes - any setting | 93 |
| Anthracyclines - any setting | 69 |
| Taxane and Anthracyclines - any setting | 67 |
| Metastatic Taxane | 57 |
| Platinum agents | 24 |
| Fluoropyrimidine agents | 78 |
| Eribulin | 33 |
| Hormonal agents for metastatic disease | 100 |
| CDK 4/6 inhibitors, % | 69 |
| mTOR inhibitor, % | 54 |
| Number of metastatic sites at study entry, % | |
| 1 | 24 |
| 2 | 38 |
| 3+ | 33 |
| Sites of metastatic disease at study entry, % | |
| Lung/mediastinum | 31 |
| Bone | 100 |
| Chest wall | 37 |
| Brain | 0 |
| Liver | 81 |

TABLE 6
Overall Response Assessment Analysis in Various
Patient Subsets from IMMU 132-01 Study

| Subject Subset | ORR, % (n/N) | |
|---|---|---|
| Overall | 31 | (17/54) |
| Age | | |
| <65 | 29 | (12/42) |
| ≥65 | 42 | (5/12) |
| Onset of metastatic disease from diagnosis | | |
| <1 year | 15 | (2/3) |
| ≥1 year | 37 | (15/41) |
| ≥2 prior chemos for metastatic disease | 29 | (12/41) |
| <2 prior chemos for metastatic disease | 38 | (5/13) |
| Prior CDK 4/6 inhibitors | 24 | (9/37) |
| No prior CDK 4/6 inhibitors | 47 | (8/17) |
| Visceral involvement at study entry (Liver/Lung) | | |
| Yes | 27 | (13/48) |
| No | 67 | (4/6) |
| Liver involvement | | |
| (More than 2 Metastates = 95%) | 27 | (12/44) |

Patients received 10 mg/kg of SG as an intravenous fusion on Days 1 and 8 of a 21-day cycle until disease progression, unacceptable toxicity, death, or withdrawal of consent. Tumor response was determined by investigator assessment according to RECIST version 1.1. Patients with a complete response (CR) or partial response (PR) were required to have repeat scans to confirm the response 4-6 weeks later.

As shown in Table 6, at a median follow-up of 11.5 months, 17/54 of the patients achieved an objective response (31.5%) [95% CI], all of which were confirmed PRs. The median PFS was 5.5 months [95% CI, 19.5%-45.6%], whereas the median OS was 12.0 months [95% CI 9.0-18.2].

In the subgroup with prior CDK 4/6 inhibitor therapy, the ORR was 25.0% (8/32; 95% CI 11.5-43.4) compared with 40.9% (9/22: 95% CI 20.7-63.6)) in patients who had no prior CDK 4/6 inhibitor therapy. Median PFS and OS in patients with no prior CDK 4/6 inhibitor therapy was 7.6 months (95% CI 5.1-10.6) and 21.7 months (95% CI 8.8-38.4) respectively, compared with patients with prior CDK 4/6 therapy [3.8 months (95% CI 1.9-6.5)] and 11.0 months [95% CI 8.1-16.4)], respectively). Limitations of this study include the small sample size and its non-randomized design. As shown, the median PFS and OS for patients with prior CDK 4/6 therapy was much lower than those that never had such therapy. The TROPiCS-02 study, on the other hand, had prior CDK4/CDK6 therapy as inclusion criteria.

Impact of biomarkers on clinical outcomes in this small population was not possible. Because only 9% of patients (n=5) in this population were homozygous for UGTIA1 allele, a safety analysis by UGTIA1 status was not performed. Trop-2 expression was not evaluated owning to limitations of small sample size. Without an active control, safety and efficacy data must be further assessed in a randomized controlled study.

### Example 2

Phase 3 Study of Sacituzumab Govitecan Versus Treatment of Physician's Choice (TPC) in Subjects with Hormonal Receptor-Positive (HR+) Human Epidermal Growth Factor Receptor (HER2) Negative Metastatic Breast Cancer (MBC) who have Failed at Least Two Prior Chemotherapy Regimens

Methods: This was an open-label, multicenter, international Phase 3 study to compare the efficacy and safety of SG versus TPC in subjects with metastatic or locally-recurrent inoperable HR+/HER2- MBC who had progressed after a CDK 4/6 inhibitor, endocrine therapy, a taxane, and at least 2, but no more than 4 prior chemotherapy regimens for metastatic disease. Subjects must have received previous endocrine therapy, taxane, and CDK4/CDK6 inhibitor therapy for inclusion in this trial.

Subjects were randomized in a 1:1 ratio to either SG (Investigational Arm A) or TPC (Control Arm B; i.e., eribulin, capecitabine, gemcitabine, or vinorelbine). Randomization was stratified based on prior chemotherapy regimens for treatment of metastatic disease (two vs. three/four lines), visceral metastasis (Y/N), and endocrine therapy in the metastatic setting for at least 6 months.

The study was conducted in two phases, a Pre-randomization Phase and a Randomization Phase.

The Pre-randomization Phase lasted no longer than 28 days and consisted of the following two periods: (1) A Screening Period to establish study eligibility and (2) A Baseline Period to confirm eligibility and establish disease characteristics prior to randomization and treatment.

The Randomization Phase began at the time of randomization of the first subject and ended on the data cut-off date for the final analysis of OS; the Randomization Phase consisted of the following two periods: (1) A Treatment Period which began at the time of randomization and ended with the completion of the End-of-Treatment (EOT) visit, which occurred at least 30 days after the final dose of study treatment and (2) A Follow-up Period which began the day after the EOT visit and continued as long as the subject was alive or until the data cut-off date of the final analysis of OS, unless the subject withdrew consent from the study or the Sponsor terminated the study.

An independent Data Safety Monitoring Committee (DSMC) was convened at regular intervals to assess the progress of this study and review safety.

### Diagnostic and Criteria for Inclusion

### Inclusion Criteria

Subjects must meet the following inclusion criteria:
(1) Female or male subjects, adult or aged ≥18 years at the time of signing the informed consent form
(2) Documented evidence of HR+/HER2- MBC confirmed by a local laboratory with the most recently available or newly obtained tumor biopsy (preferably within the last 12 months) from a locally recurrent or metastatic site(s) and defined per American Society of Clinical Oncologists/College of American Pathologists criteria as:
   a. HR+ (a tumor is considered HR+ if at least 1% of the cells examined have estrogen and/or progesterone receptors)
   b. HER2- defined as immunohistochemistry ≤2+ or fluorescence in situ hybridization negative
(3) Availability of archival tumor tissue in a formalin fixed, paraffin embedded (FFPE) block (preferably within 12 months prior to consent) or newly acquired biopsy (FFPE block) from a metastatic site. Note: Bone biopsies are not allowed.
(4) Refractory to or relapsed after at least 2, but no more than 4 prior systemic chemotherapy regimens for metastatic disease. Adjuvant or neoadjuvant therapy for early stage disease will qualify as one of the required prior chemotherapy regimens if the development of unresectable, locally advanced, or metastatic disease occurred within a 12-month period of time of the therapy. Note: Treatments for bone metastases (e.g., bisphosphonates, denosumab, etc.) and hormonal therapy are not considered as prior systemic chemotherapy treatments for advanced disease.
(5) Should have been previously treated with:
   At least 1 taxane in any setting
   At least 1 prior anticancer hormonal treatment in any setting
   At least 1 CDK 4/6 inhibitor in any setting
(6) Eligible for one of the chemotherapy options listed in the TPC arm
(7) Documented disease progression after the most recent therapy by computed tomography (CT)/magnetic resonance imaging (MRI)
(8) At least 1 measurable target lesion according to RECIST 1.1 (bony disease only is not allowed) that meets all of the following criteria:
   a. Lymph node lesion that measures at least ≥1.5 cm in the short axis
   b. Non-nodal lesion that measures ≥1.0 cm in the longest diameter in the plane of measurement
   c. The lesion is suitable for repeat measurement using CT/MRI. Historical CT/MRI scans performed within 28 days of CIDI may be used as screening scans to demonstrate eligibility as long as they meet minimum standards as separately defined by the central imaging vendor.
   d. Lesions that have had external beam radiotherapy or locoregional therapy must show radiographic evidence of disease progression based on RECIST 1.1 to be deemed a target lesion.
   e. Brain CT/MRI must be conducted for subjects with a history of brain metastasis. The subject must have had stable* brain metastasis for at least 4 weeks. Target lesions cannot be from brain.
* Stable brain metastasis is defined as the following:
   i. Prior local treatment by radiation, surgery, or stereotactic surgery
   ii. Imaging—stable or decreasing size after such local treatment
   iii. Clinically stable signs and symptoms for at least 4 weeks
   iv. ≥2 weeks from discontinuation of antiseizure medication
   v. Low and stable doses of corticosteroids ≤20 mg prednisone or equivalent daily are permitted
(9) Eastern Cooperative Oncology Group (ECOG) performance status of 0 or 1
(10) Adequate renal function: calculated creatinine clearance ≥30 mL/minute according to the Cockcroft and Gault formula
(11) Adequate bone marrow function, defined as:
   Absolute neutrophil count (ANC)≥1,500 per mm3
   Hemoglobin≥9.0 g/dL
   Platelet count≥100,000 per mm3
   Note: Blood transfusion or growth factor support is not allowed within 14 days prior to screening labs.
(12) Adequate liver function, defined as:
   Total bilirubin≤1.5× institutional upper limit of normal (IULN) or ≤3 IULN for patients with documented Gilbert's syndrome
   Alanine aminotransferase (ALT), and aspartate aminotransferase (AST)≤2.5× IULN (in the case of liver metastases≤5× IULN), and serum albumin≥3 g/dL
   Alkaline phosphatase (ALP)≤5.0× IULN unless there are bone metastases, in which case liver-specific ALP must be separated from the total and used to assess liver function instead of total ALP
(13) Resolution of all systemic anticancer therapy-related or radiation-related toxicities to Grade I severity or lower, except for neuropathy (≤Grade 2) and alopecia. Subjects with Grade 2 neuropathy are eligible, but should not receive vinorelbine as TPC.
(14) Females must not be lactating or pregnant at Baseline (as documented by a negative beta human chorionic gonadotropin [β-hCG] or human chorionic gonadotropin [hCG] test with a minimum sensitivity of 25 IU/L or equivalent units of β-hCG [or hCG]). All females will be considered to be of childbearing potential unless they are postmenopausal (amenorrhoeic for at least 12 consecutive months, in the appropriate age group, and without other known or suspected cause) or have been sterilized surgically (i.e., bilateral tubal ligation, total hysterectomy, or bilateral oophorectomy, all with surgery at least 1 month before dosing).
(15) Females of childbearing potential must not have had unprotected sexual intercourse within 30 days before study entry and must agree to use a highly effective method of contraception (total abstinence [if it is her preferred and usual lifestyle], a contraceptive implant, an oral contraceptive, or have a vasectomized partner with confirmed azoospermia) throughout the entire study period and for 6 months after study drug discontinuation. For sites outside of the European Union (EU), it is permissible that if a highly effective method of contraception is not appropriate or acceptable to the subject, then the subject must agree to use a medically acceptable method of contraception, i.e., double barrier methods of contraception such as condom plus diaphragm or cervical/vault cap with spermicide. If currently abstinent, the subject must agree to use a highly effective method as described above if she becomes sexually active during the study period or for 6 months after study drug discontinuation. Females who are using hormonal contraceptives must have been on a stable dose of the same hormonal contraceptive product for at least 28 days before dosing and must continue to use the same contraceptive during the study and for 6 months after study drug discontinuation.
(16) Male subjects who are partners of women of childbearing potential must use a condom and spermicide and their female partners, if of childbearing potential, must use a highly effective method of contraception (see methods described above in Inclusion Criterion 15) beginning at least 1 menstrual cycle prior to starting study drug, throughout the entire study period, and for 3 months after the last dose of study drug, unless the male subjects are totally sexually abstinent or have undergone a successful vasectomy with confirmed azoospermia or unless the female partners have been sterilized surgically or are otherwise proven sterile.
(17) Must be willing and able to comply with all aspects of the protocol
(18) Must voluntarily agree to provide written informed consent
(19) Could have received an unlimited number of prior endocrine, biological, or targeted therapies in the absence of co-administered chemotherapy: all of these therapies must have been completed 14 days prior to randomization, except biological therapy which must have been completed 28 days prior to randomization

### Exclusion Criteria

Subjects who meet any of the following criteria will be excluded from the study:
(1) Previous treatment with a topoisomerase 1 inhibitor as a free form or as other formulations
(2) Current enrollment in another clinical study or used any investigational device or drug either within 5 half-lives or 28 days prior to randomization, whichever is longer
(3) Treatment with chemotherapy, radiation, or small molecule targeted therapy within 2 weeks and biological therapy within 4 weeks prior to the first dose of study treatment
(4) Existing anticancer treatment-related adverse events (AEs) of Grade≥2 (except for alopecia and Grade 2 neuropathy) according to National Cancer Institute (NCI)-CTCAE v5.0
(5) Any other malignancy that required treatment or has shown evidence of recurrence (except for non-melanoma skin cancer or histologically-confirmed complete excision of carcinoma in situ) during the 5 years prior to enrollment in this study
(6) History of significant cardiovascular disease, defined as:
   Congestive heart failure greater than New York Heart Association (NYHA) Class II according to the NYHA Functional Classification
   Unstable angina or myocardial infarction within 6 months before enrollment
   Serious cardiac arrhythmia
(7) Clinically-significant electrocardiogram (ECG) abnormality, including any of the following:
   Marked Baseline prolonged QT/QTc interval (i.e., a repeated demonstration of a QTc interval>500 ms) demonstrated on ECG at Screening.
      QTcF is calculated by Fridericia's formula
   History of risk factors for torsade de pointes (e.g., heart failure, hypokalemia, family history of long QT Syndrome)
(8) Has known active central nervous system metastases and/or carcinomatous meningitis. Subjects may participate provided they have stable brain metastasis. All subjects with carcinomatous meningitis are excluded regardless of clinical stability. Stable brain metastasis is defined in inclusion criterion 8
(9) Active hepatitis B virus (positive hepatitis B surface antigen) or active hepatitis C virus (measurable viral ribonucleic acid (RNA) load with polymerase chain reaction) infection
(10) Scheduled surgery during the study, other than minor surgery which would not delay study treatment
(11) Has an active serious infection requiring antibiotics
(12) Has active chronic inflammatory bowel disease (ulcerative colitis, Crohn's disease) and subjects with a history of bowel obstruction
(13) Have received a live vaccine within 30 days of randomization
(14) Known hypersensitivity or intolerance to any of the study drugs or any of the excipients
(15) Any medical or other condition which, in the opinion of the Investigator, causes the subject to be medically unfit to receive sacituzumab govitecan or unsuitable for any other reason
(16) Is receiving any medication prohibited in combination with the study treatment(s) as described in the respective product labels, unless medication was stopped within 7 days prior to randomization
(17) Locally-advanced MBC (stage IIIc) in subjects who are candidates for curative intent therapy at the time of study enrollment.
   If required per local guidelines, any subject with a blood uracil level≥150 ng/ml is excluded from receiving capecitabine as TPC (Note: blood uracil level will be assessed at Screening for all subjects eligible to be randomized to capecitabine as TPC)

### Investigational Product, Dosage and Mode of Administration

Sacituzumab govitecan 10 mg/kg administered as an intravenous (IV) infusion on Days 1 and 8 of repeating 21-day cycles. Protocol-defined dose reductions were permitted. Subjects in this group will receive pre-medications (i.e., antipyretics, H₁ blockers, and H₂ blockers) for prevention of infusion reactions and a 2- or 3-drug combination regimen for prevention and treatment of chemotherapy-induced nausea, vomiting, and diarrhea.

### Reference Therapy, Dosage and Mode of Administration

TPC with one of the following:
Eribulin—(1.4 mg/m² for North American sites, 1.23 mg/m² for EU sites or per institution) is to be administered IV on Days 1 and 8 of a 21-day cycle
Capecitabine—(1000-1250 mg/m²) is to be administered PO twice daily for 2 weeks followed by a 1-week rest period given as a 21-day cycle
Gemcitabine—(800-1200 mg/m²) administered IV on Days 1, 8, and 15 of each 28-day cycle or per institution
Vinorelbine (25 mg/m² IV on Day I weekly cycle per institution) (Note: subjects with grade 2 neuropathy are eligible, but should not receive vinorelbine as TPC)
The use of pre-medications (i.e., antipyretics, H₁ blockers, and H₂ blockers) for prevention of infusion reactions and medications for prevention and treatment of chemotherapy-induced nausea, vomiting, and diarrhea for subjects in this group is based on the Investigator's discretion.

### Duration of Treatment

Subjects continued to receive study treatment until RECIST 1.1 defined progressive disease (PD) by LIR, development of unacceptable toxicity, subject request, withdrawal of consent, Investigator decision, pregnancy, or study termination by the Sponsor, or another treatment discontinuation criteria is met.

### Criteria for Evaluation

### Efficacy

Efficacy analyses was performed using tumor assessments by LIR and BICR using RECIST 1.1.

### Primary Efficacy End Point

PFS as determined by BICR using RECIST 1.1

### Secondary Efficacy End Points

OS
ORR as determined by BICR using RECIST 1.1
TTD in the global health status/QOL, pain and fatigue domains of EORTC QLQ-C30
DOR as determined by LIR and BICR using RECIST 1.1
CBR as determined by LIR and BICR using RECIST 1.1

### Statistical Methods: Statistical Methods

### Sample Size Assumptions

The sample size was estimated based on the primary end point of PFS, but also took into consideration OS as the main secondary end point. An overall sample size of approximately 520 subjects were randomized in a 1:1 ratio to either sacituzumab govitecan or TPC.

For PFS, assuming a hazard ratio of 0.70 (medians of 5.3 months for sacituzumab govitecan and 3.7 months for TPC), a total of 350 PFS events were used to detect a statistically significant difference at a 2-sided alpha of 0.05 with 92% power. With an estimated average accrual rate of 22 subjects per month, a total of 520 subjects provided 350 PFS events approximately 27 months after the first subject is randomized, after accounting for events being censored because of subjects missing tumor assessments or starting subsequent anti-cancer therapies. The recruitment rate was assumed to be non-uniform so that half of the subjects were recruited 55% of the way through the recruitment period of approximately 24 months reflecting the change in sample size and actual recruitment rate that was affected by the global coronavirus disease 2019 (COVID-19) pandemic.

At PFS final analysis. OS was summarized descriptively only. To be conservative, the nominal 2-sided alpha of 0.00001 will be spent even without formal hypothesis testing. For OS. assuming a hazard ratio of 0).73 (medians of 16.5 months in Arm A and 12 months in Arm B), a total of 438 OS events were needed to detect a statistically significant difference with 86.7% power at a 2-sided alpha of 0.04999 based on a recruitment period of approximately 24 months and 52 months of survival follow-up from the first subject randomized.

The Sponsor closely monitored the number of subjects randomized and discontinued. including subjects who refused study treatment assigned. As the primary analysis was triggered by a targeted number of PFS events, subjects who prematurely discontinued from the study or whose events were censored did not count toward the targeted number. To compensate for such cases. an additional number of subjects was necessary to be enrolled to ensure the targeted number of events was reached within a reasonable timeframe.

### Multiplicity Adjustment

The overall type I error rate for this study was strictly controlled at a 2-sided alpha of 0.05. The primary end point analysis of PFS assessed by BICR served as the gatekeeper for the secondary end point analyses and was tested at the 2-sided alpha of 0.05. At PFS final analysis. OS was summarized descriptively only. To be conservative. the nominal 2-sided alpha of 0.00001 was spent even without formal hypothesis testing. Since the primary PFS analysis was positive. analysis of the main secondary end point of OS was formally tested sequentially at the 2-sided alpha of 0.04999. ORR (assessed by BICR) and analysis for QOL was formally tested sequentially at the 2-sided alpha of 0.05 respectively. For analysis of QOL. TTD of global health status/QOL. pain. and fatigue domains as measured by EORTC QLQ-C30 were tested.

### Primary End Point (Primary Analysis)

PFS was described using Kaplan-Meier (K-M) estimates. The primary analysis of PFS for the comparison between treatment arms was performed using a stratified log rank test with the stratification factors used in the randomization. Median PFS and its 95% CI was determined by the Brookmeyer and Crowley method with log-log transformation was presented and the K-M estimates of PFS were plotted over time. Hazard ratio of PFS and its 95% CI were estimated using Cox proportional-hazards model stratified by the same stratification factors used in the randomization.

### Secondary End Points (Secondary Analyses)

OS was described using K-M estimates. The primary analysis of OS for comparison between treatment arms was performed using a stratified log rank test with the same stratification factors used in the randomization. Median OS and the associated 95% CI as determined by the Brookmeyer and Crowley method with log-log transformation was presented. Hazard ratio and the associated 95% CI was estimated using a Cox proportional-hazards model stratified by the same stratification factors used in the randomization.

ORR was analyzed and compared between the treatment arms using the Cochran Mantel Haenszel (CMH) test stratified by the stratification factors used in the randomization. The 2-sided 95% CIs was calculated using the Clopper-Pearson exact method.

CBR was calculated with exact 95% CIs using the method of Clopper and Pearson. CBR was compared between treatment arms using a CMH test stratified by the stratification factors used in the randomization. The differences and odds ratios of these rates between treatment arms and 95% CIs was calculated respectively.

The K-M estimates of median DOR and its 95% CI was calculated for responders (CR or PR) in each treatment arm.

Time to deterioration of global health status/QOL, pain, and fatigue domains as measured by EORTC QLQ-C30 was analyzed similarly as the primary analysis of PFS.

TABLE 7
Demographics and Baseline Characteristics of
Patients in the TROPiCS-02 Clinical Trial

| | SG (n = 272) | TPC (n = 271) |
|---|---|---|
| Female, % | 99 | 99 |
| Median age, (range) y | 57 (29-86) | 55 (27-78) |
| <65 y, % | 73 | 75 |
| ≥65 y, % | 27 | 25 |
| Race or ethnic group, % | | |
| White | 68 | 66 |
| Black | 3 | 5 |
| Asian | 4 | 2 |
| Other^{a}/Not reported^{b} | 25 | 28 |
| Geographic region, % | | |
| North America | 42 | 42 |
| Europe | 58 | 58 |
| ECOG PS, % | | |
| 0 | 43 | 46 |
| 1 | 57 | 54 |
| Visceral metastases at baseline, % | 95 | 95 |
| Liver metastases,^{c} % | 84 | 87 |
| De novo metastatic breast cancer, % | 29 | 22 |
| Median time from initial metastatic | 48.5 | 46.6 |
| diagnosis to randomization, (range) mo | (1.2-243.8) | (3.0-248.8) |
| Prior chemotherapy in (neo)adjuvant | 64 | 68 |
| setting, % | | |
| DFI <12 mo, % | 8 | 8 |
| Prior endocrine therapy use in the | 86 | 86 |
| metastatic setting ≥6 mo, % | | |
| Prior CDK4/6 inhibitor use, % | | |
| ≤12 months | 59 | 61 |
| >12 months | 39 | 38 |
| Unknown | 2 | 1 |
| Number of prior lines of chemotherapy, % | | |
| ≤2 | 42 | 44 |
| ≥3 | 58 | 56 |
| Median prior chemotherapy regimens in | 3 (0-8) | 3 (1-5) |
| the metastatic setting, n (range)^{d} | | |

Results from TROPiCS-02 Clinical Trial

A total of 543 patients were enrolled and randomly assigned to the SG group (272 patients) or the control TPC group (271 patients). Table 6 shows the demographics and baseline characteristics of the subjects in the TROPiCS-02 clinical trial.

1^{st} OS Analysis: On Jun. 4, 2022, results from the primary analysis (at the one-year mark) of the Ph 3 TROPiCS-02 study of SG versus TPC were announced. The primary end point of PFS survival was met (SG treatment: 5.5 months; TPC: 4.0 months; HR: 0.66; 95% CI: 053-0.83; P<0.0003); however, due to the slim survival benefit (1.5 months) it was unclear whether, at the first interim analysis, the results were clinically relevant. The difference in OS between SG versus TPC, however, was not considered statistically significant nor were they considered clinically relevant (SG: 13.9 months and TPC: 12.3 months; hazard ratio of 0.84 and p value of 0.143) at the time.

2^{nd} interim OS analysis-The results of the analysis are shown in Table 8, below. SG demonstrated statistically significant improvement in PFS and OS versus TPC. The OS data is based on 390 events.

TABLE 8
2^{nd} Interim Analysis - TROPiCS-02 Clinical Trial
Overall Survival³

| | | |
|---|---|---|
| Median OS in months | 14.4 | 11.2 |
| (95% CI) | (13.0, 15.7) | (10.1, 12.7) |

| | |
|---|---|
| Hazard ratio (95% CI) | 0.789 (0.646, 0.964) |
| p-value² | 0.0200 |

Objective Response Rate³ by BICR

| | | |
|---|---|---|
| Response Rate, % (95% CI) | 21.0 | 14.0 |
| | (16.3, 26.3) | (10.1, 18.7) |

| | |
|---|---|
| Odds ratio (95% CI) | 1.625 (1.034, 2.555) |
| p-value | 0.0348 |

Duration of Response³ (DOR) by BICR

| | | |
|---|---|---|
| Median DOR in months | 8.1 | 5.6 |
| (95% CI) | (6.7, 9.1) | (3.8, 7.9) |

Final Results from TROPiCS-02 Clinical Trial

Additional efficacy analysis from TROPiCS-02 with a longer median follow-up of about 12.75 months (data cutoff Dec. 1, 2022) are detailed below and shown in FIGS. 2-7; 9-12. As shown in FIG. 2, SG continued to demonstrate improvement in PFS versus TPC at a longer follow-up, with a 35% reduction in risk. As shown in FIG. 3, SG continued to demonstrate an improvement in OS vs. TPC, with a 21% reduction in death, and a higher proportion of patients that remained alive at each landmark. The median OS was 14.5 months for SG, and 11.2 months for TPC.

As shown in FIGS. 4-5, treatment with SG resulted in greater PFS regardless of HER2 IHC status. In patients with HR+/HER2-low breast cancer (FIG. 4), median PFS was greater in SG-treated patients (SG: 5.8 months, TPC: 4.2 months). In patients with HR+/HER2- IHC0 breast cancer (FIG. 5), median PFS was greater in SG-treated patients (SG: 5.0 months, TPC: 3.4 months).

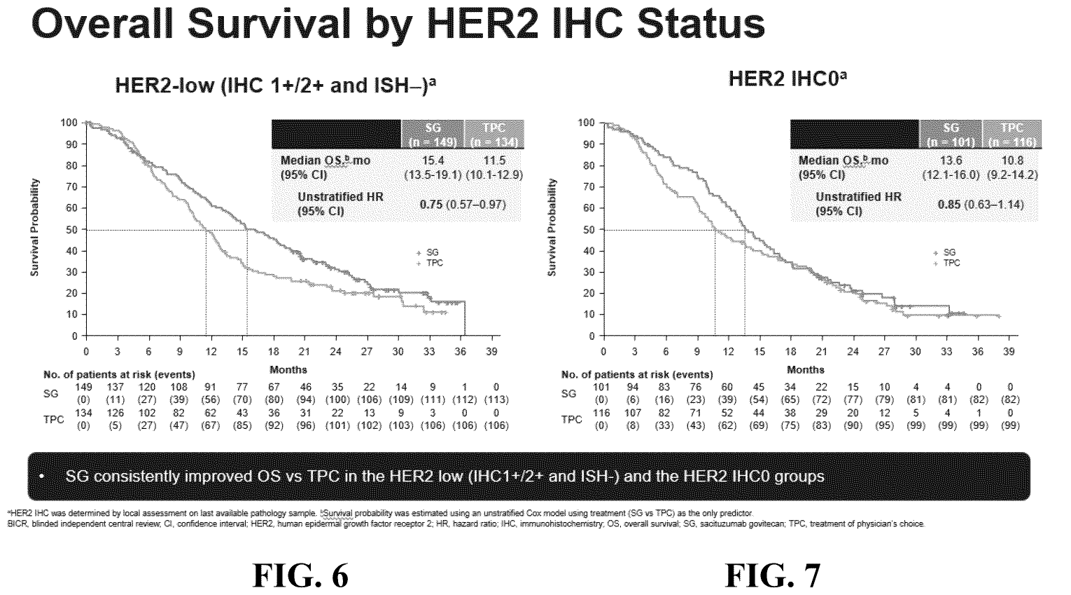

As shown in FIGS. 6-7, treatment with SG resulted in greater OS regardless of HER2 IHC status. In patients with HR+/HER2-low breast cancer (FIG. 6), median OS was greater in SG-treated patients (SG: 15.4 months, TPC: 11.5 months). In patients with HR+/HER2- IHC0 breast cancer (FIG. 7), median OS was greater in SG-treated patients (SG: 13.6 months, TPC 10.8 months).

### Example 3

Trop-2 mRNA Expression and Association with Clinical Outcomes with SG in Patients with HR+/HER2- metastatic Breast Cancer (mBC): Biomarker Results from the Phase 2 TROPiCS-02 Study

The objective of this study was to measure the efficacy outcomes for SG versus TPC by Trop-2 gene (TACSTD2) mRNA expression and the correlation of TACSTD2 expression with other genes.

RNA from patients in the TROPiCS-02 study was isolated from archival tumor tissue samples using AllPrep DNA/RNA kits (Qiagen) and libraries prepared with TruSeq RNA Exome Prep Kit (Illumina). Sequencing was via NovaSeq (2X150bp) (Illumina). Gene expression quantitation was performed using Salmon. TACSTD2 expression was defined as high or low via medium cut.

In the mRNA analytical cohort (N=197), 49% had TACSTD high expression (SG, n=51; TPC, n=46), and 51% had TACSTD2 low expression (SG, n=47; TPC, n=53). A positive concordance between TACSTD2 expression and Trop-2 IHC (H-Score) was observed (categorical concordance 71%; Cohen's kappa=0.41).

TACTD2 expression was similar across HR+/HER2 HER+/IHC0 and HR+/HER2-Low subgroups [log2 transcripts per million (TPM) of 3.36 (IQR, 2.76-4.22) and log2 TPM of 3.44 (IQR, 2.59-4.22)], respectively. SG showed a numerically higher median PFS [TACSTD2 high: SG (7.3 months); TPC (5.6 months); TACSTD2 low: SG (5.6 months); TPC 2.8 months)] versus TPC regardless of TACSTD2 expression. Similarly, for OS, outcomes favored SG over TPC regardless of TACSTD2 expression.

This mRNA-based biomarker analysis demonstrated that SG improves PFS and OS outcomes in patients with pretreated, endocrine-resistant HR+/HER2- metastatic breast cancer regardless of Trop-2 mRNA expression.

### Example 4

Safety outcomes by UGT1A1 status in the phase 3 TROPiCS-02 study of SG in HR+/HER2- Metastatic Breast Cancer

Increased Risk of Adverse Reactions in Patients with Reduced UGT1A1 Activity

The UGT1A1 gene encodes UDP-glucuronosyltransferase (“UGT”). A common cause of decreased UGT1A1 activity is the insertion of a TA in the TATA box at the promoter region of the UGT1A1 gene, which was named UGT1A1*28. SN-38 is inactivated and detoxified by UDP-glucuronosyltransferase. The risk of irinotecan toxicity increases with genetic variants associated with reduced UGT enzyme activity, such as UGT1A1*28 allele. Neutropenia is more likely to develop in subjects homozygous or heterozygous for the mutation.

A post-hoc safety analysis was performed by UGT1A1 status in the TROPiCS-02 study trial. Of the 543 enrolled patients, 517 received≥1 dose of study treatment. UGT1A1 status in the SG cohort was n=104 (38%, wild type): n=119 (44%, heterozygous): and n=25 (9% homozygous *28/*28).

In SG patients, grade≥3 TEAEs included neutropenia: (52%), diarrhea: (10%), anemia: (8%), and febrile neutropenia: (6%). Specifically, incidence of these TEAEs by UGT1A1 status (*1/*1; *1/*28; and *28/*28) was neutropenia: 45%, 57%, and 64%; diarrhea 6%, 13%, and 24%; anemia (6%, 6%, and 8%); febrile neutropenia (6%, 7%, and 4%); respectively.

### Example 5

### TROPiCS-02 Data in HR+/HER2- Metastatic Breast Cancer Patients Demonstrates Progression-Free Survival and Overall Survival Benefit of Trodelvy Regardless of Their HER2 Status

The analysis examined PFS and OS by HER2-immunohistochemisty (IHC) status, and the results demonstrated that SG improved median PFS and OS vs. TPC in both HER2-low (IHC 1+ and IHC 2+/ISH-negative) and IHCO groups.

Patients were tested for HER2 protein expression by IHC and/or gene expression by in situ hybridization (ISH). HER2-low breast cancer is a newly defined subset of HER2-negative breast cancer that has a HER2 immunohistochemical score of 1+ or score of 2+/in situ hybridization (ISH) negative phenotype. Breast cancers are classified as HER2 positive when (1) HER2 expression is scored 3+ by IHC or (2) an IHC score of 2+ with HER2 gene amplification tested by ISH. Breast cancers with HER2 IHC score of 0 or 1+, or an IHC score of 2+ without gene amplification are considered as HER2-negative.

Summary of the results are shown in Tables 8a-8d.

### Tables 8a and 8b: Progression-Free Survival and HER2 Status

TABLE 8a
PFS in HR+/HER2− low breast cancer

| BICR analysis | SG (n = 149) | TPC (n = 134) |
|---|---|---|
| Median PFS,^{b} | 5.8 | 4.2 |
| mo (95% CI) | (4.1-8.4) | (2.8-4.5) |
| Unstratified HR | 0.60 (0.44-0.82) | |
| (95% CI) | | |

TABLE 8b
PFS in HR+/HER2− IHC0 breast cancer

| BICR analysis | SG (n = 101) | TPC (n = 116) |
|---|---|---|
| Median PFS,^{b} | 5.0 | 3.4 |
| mo (95% CI) | (3.9-7.2) | (1.8-4.2) |
| Unstratified HR | 0.70 (0.51-0.98) | |
| (95% CI) | | |

TABLE 8c
OS in HR+/HER2− low breast cancer

| | SG (n = 149) | TPC (n = 134) |
|---|---|---|
| Median OS,^{b} mo | 15.4 | 11.5 |
| (95% CI) | (13.5-19.1) | (10.1-12.9) |
| Unstratified HR | 0.75 (0.57-0.97) | |
| (95% CI) | | |

TABLE 8d
OS in HR+/HER2− IHCO breast cancer

| | SG (n = 101) | TPC (n = 116) |
|---|---|---|
| Median OS.^{b} mo | 13.6 | 10.8 |
| (95% CI) | (12.1-16.0) | (9.2-14.2) |
| Unstratified HR | 0.85 (0.63-1.14) | |
| (95% CI) | | |

The data demonstrated SG's efficacy across HER2-low and ICH0 status compared to TPC in pre-treated metastatic breast cancer in the TROPiCS-02 trial.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating HR+/HER2- breast cancer in a population of human subjects that is statistically significantly superior to a treatment of physician's choice (TPC), comprising administering to the human subjects sacituzumab govitecan (SG), wherein the human subjects previously received an endocrine-based therapy and at least two additional systemic therapies, wherein a dosage of 10 mg/kg of SG is administered as an intravenous infusion once weekly on day 1 and day 8 of one or more 21-day treatment cycles, and wherein the method achieves a statistically significantly greater overall survival (OS) compared to a control group of human subjects treated with the TPC.

2. The method of claim 1, wherein the method achieves a median OS of 14.5 months in the population of human subjects treated with SG and a median OS of 11.2 months in the control group of human subjects treated with the TPC at a median follow-up of about 12.75 months.

3. The method of claim 1, wherein at least one of the at least two additional systemic therapies was administered in the metastatic setting.

4. The method of claim 1, wherein at least one of the at least two additional systemic therapies comprises a chemotherapy.

5. The method of claim 4, wherein the chemotherapy comprises a taxane.

6. The method of claim 1, wherein the human subject previously received three additional systemic therapies.

7. The method of claim 1, wherein the human subject previously received four additional systemic therapies.

8. The method of claim 1, comprising administering SG for about 8 cycles or more.

9. The method of claim 8, comprising administering SG for about 11 cycles or more.

10. The method of claim 1, wherein the human patient has HR+/HER2-IHC0 breast cancer.

11. The method of claim 1, wherein the human patient has HR+/HER2- IHC1+ breast cancer.

12. The method of claim 1, wherein the human patient has HR+/HER2- IHC2+/ISH-breast cancer.


SEQUENCE LISTING (20 sequences)

SEQ ID NO: 1 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3-12 - skipped (10 entries)

SEQ ID NO: 13 - protein, 5 aa, synthetic construct
NYGMN

SEQ ID NO: 14 - protein, 17 aa, synthetic construct
WINTYTGEPTYTDDFKG

SEQ ID NO: 15 - protein, 12 aa, synthetic construct
GGFGSSYWYFDV

SEQ ID NO: 16 - protein, 11 aa, synthetic construct
KASQDVSIAVA

SEQ ID NO: 17 - protein, 7 aa, synthetic construct
SASYRYT

SEQ ID NO: 18 - protein, 9 aa, synthetic construct
QQHYITPLT

SEQ ID NO: 19 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 20 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR